SEQUENCE LISTING (16 sequences)

SEQ ID NO: 1 - protein, 102 aa, Artificial Sequence
  Synthetic
ATPAPKSCPERHYWAQGKLCCQMCEPGTFLVKDCDQHRKAAQCDPCIPGVSFSPDHHTRPHCESCRHCNSGLLVRNCTITANAECACRNGWQCRDKECTECD

SEQ ID NO: 2 - protein, 346 aa, Artificial Sequence
  Synthetic
ATPAPKSCPERHYWAQGKLCCQMCEPGTFLVKDCDQHRKAAQCDPCIPGVSFSPDHHTRPHCESCRHCNSGLLVRNCTITANAECACRNGWQCRDKECTECDPLPNPSLTARSSQALSPHPQPTHLPYVSEMLEARTAGHMQTLADFRQLPARTLSTHWPPQRSLCSSDFIRILVIFSGMFLVFTLAGALFLKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 3 - protein, 356 aa, Artificial Sequence
  Synthetic
EQKLISEEDLATPAPKSCPERHYWAQGKLCCQMCEPGTFLVKDCDQHRKAAQCDPCIPGVSFSPDHHTRPHCESCRHCNSGLLVRNCTITANAECACRNGWQCRDKECTECDPLPNPSLTARSSQALSPHPQPTHLPYVSEMLEARTAGHMQTLADFRQLPARTLSTHWPPQRSLCSSDFIRILVIFSGMFLVFTLAGALFLKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 4 - protein, 325 aa, Artificial Sequence
  Synthetic
ATPAPKSCPERHYWAQGKLCCQMCEPGTFLVKDCDQHRKAAQCDPCIPGVSFSPDHHTRPHCESCRHCNSGLLVRNCTITANAECACRNGWQCRDKECTECDTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 5 - protein, 344 aa, Artificial Sequence
  Synthetic
ATPAPKSCPERHYWAQGKLCCQMCEPGTFLVKDCDQHRKAAQCDPCIPGVSFSPDHHTRPHCESCRHCNSGLLVRNCTITANAECACRNGWQCRDKECTECDGGGSGGGSGGGSGGGSGGGSGGGSGGGSGGGSGGGSGGGSGGGSGGGSGGGSGGGSGGGSGGGSGGGSILVIFSGMFLVFTLAGALFLKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 6 - protein, 296 aa, Artificial Sequence
  Synthetic
ATPAPKSCPERHYWAQGKLCCQMCEPGTFLVKDCDQHRKAAQCDPCIPGVSFSPDHHTRPHCESCRHCNSGLLVRNCTITANAECACRNGWQCRDKECTECDPLPNPSLTARSSLCSSDFIRILVIFSGMFLVFTLAGALFLKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 7 - protein, 276 aa, Artificial Sequence
  Synthetic
ATPAPKSCPERHYWAQGKLCCQMCEPGTFLVKDCDQHRKAAQCDPCIPGVSFSPDHHTRPHCESCRHCNSGLLVRNCTITANAECACRNGWQCRDKECTECDILVIFSGMFLVFTLAGALFLKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 8 - protein, 260 aa, Artificial Sequence
  Synthetic
MARPHPWWLCVLGTLVGLSATPAPKSCPERHYWAQGKLCCQMCEPGTFLVKDCDQHRKAAQCDPCIPGVSFSPDHHTRPHCESCRHCNSGLLVRNCTITANAECACRNGWQCRDKECTECDPLPNPSLTARSSQALSPHPQPTHLPYVSEMLEARTAGHMQTLADFRQLPARTLSTHWPPQRSLCSSDFIRILVIFSGMFLVFTLAGALFLHQRRKYRSNKGESPVEPAEPCHYSCPREEEGSTIPIQEDYRKPEPACSP

SEQ ID NO: 9 - protein, 12 aa, Artificial Sequence
  Synthetic
TRPHCESCRHCN

SEQ ID NO: 10 - protein, 20 aa, Artificial Sequence
  Synthetic
ILVIFSGMFLVFTLAGALFL

SEQ ID NO: 11 - protein, 112 aa, Artificial Sequence
  Synthetic
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 12 - protein, 42 aa, Artificial Sequence
  Synthetic
KRGRKKLLYIFKQPFMRPVQTTQEEDGCSCRFPEEEEGGCEL

SEQ ID NO: 13 - protein, 70 aa, Artificial Sequence
  Synthetic
PLPNPSLTARSSQALSPHPQPTHLPYVSEMLEARTAGHMQTLADFRQLPARTLSTHWPPQRSLCSSDFIR

SEQ ID NO: 14 - protein, 19 aa, Artificial Sequence
  Synthetic
MARPHPWWLCVLGTLVGLS

SEQ ID NO: 15 - protein, 21 aa, Artificial Sequence
  Synthetic
MALPVTALLLPLALLLHAARP

SEQ ID NO: 16 - protein, 10 aa, Artificial Sequence
  Synthetic
EQKLISEEDL

(19) United States
(12) Patent Application Publication
(10) Pub. No.: US 2022/0411478 A1
(43) Pub. Date: Dec. 29, 2022

(54) CD70 TARGETED CHIMERIC ANTIGEN RECEPTOR (CAR) T CELLS AND USES THEREOF

(71) Applicant: The General Hospital Corporation, Boston, MA (US)

(72) Inventors: Marcela V. Maus, Lexington, MA (US); Mark Leick, Boston, MA (US)

(73) Assignee: The General Hospital Corporation, Boston, MA (US)

(21) Appl. No.: 17/761,131

(22) PCT Filed: Sep. 16, 2020
(86) PCT No.: PCT/US2020/051018

§ 371 Date: Mar. 16, 2022

Related U.S. Application Data
(60) Provisional application No. 62/900,826, filed on Sep. 16, 2019.

(51) Int. Cl. C07K 14/725; C07K 16/28; C12N 15/864; A61P 35/02

(52) U.S. Cl. CPC C07K 14/7051; C07K 16/2875; C07K 16/2878; C12N 15/8645; A61P 35/02

(57) ABSTRACT

Provided herein are CD70 targeting chimeric antigen receptors and engineered immune cells (e.g., T cells) comprising such CAR. Method of treating a cancer expressing CD70 using such engineered immune cells are also provided. In some embodiments, the method of treating cancer further comprising using an agent that enhances CD70 expression in the cancer (e.g., azacitidine) in combination with the engineered immune cells comprising the CD70-targeting CAR.

### RELATED APPLICATION

This application claims the benefit under 35 U.S.C. § 119(e) to U.S. Provisional Application No. 62/900,826, filed Sep. 16, 2019, entitled “Use of CD70 Targeted Chimeric Antigen Receptor (CAR) T Cells for the Treatment of Acute Myeloid Leukemia (AML),” the entire contents of each of which are incorporated herein by reference.

### GOVERNMENT SUPPORT

This invention was made with government support under Grant No. T32CA071345-21A1, awarded by The National Institutes of Health. The government has certain rights in the invention.

### BACKGROUND

CAR-T cells have led to a revolution in the treatment of advanced hematologic malignancies. Finding targets that express in myeloid malignancies but not in normal human tissues has been challenging.

### SUMMARY

The present disclosure, in some aspects, provides T-cells expressing a chimeric antigen receptor (CAR) targeting CD70 and uses of such T-cells for treating hematologic malignancies (e.g., acute myeloid leukemia (AML)). In some aspects, the present disclosure demonstrates that, surprisingly, CD-70-targeting CART-cells in combination with an agent that enhances CD70 expression in cancer cells (e.g., azacitidine) are synergistic for the treatment of AML.

Accordingly, some aspects of the present disclosure provide chimeric antigen receptors (CARs) comprising: (i) an extracellular target binding domain comprising a polypeptide that binds CD70; (ii) a transmembrane domain; and (iii) an intracellular signaling domain.

In some embodiments, the polypeptide comprises a CD70-binding domain of CD27. In some embodiments, the polypeptide comprises the extracellular domain of CD27. In some embodiments, the polypeptide comprises an amino acid sequence that is at least 80% identical to the amino acid sequence of SEQ ID NO: 1. In some embodiments, the polypeptide comprises the amino acid sequence of SEQ ID NO: 1. In some embodiments, the polypeptide comprises an anti-CD70 antibody, optionally an scFv. In some embodiments, the transmembrane domain is the transmembrane domain of CD27. In some embodiments, the intracellular signaling domain comprises (i) an ITAM-containing signaling domains and/or (ii) one or more signaling domains from one or more co-stimulatory proteins or cytokine receptors. In some embodiments, the intracellular signaling domain comprises a CD3γ, CD3ε, CD3δ or CD3ζ. In some embodiments, the intracellular signaling domain comprises CD3. In some embodiments, the costimulatory domain comprises CD28, 41BB, 2B4, KIR, OX40, ICOS, MYD88, IL2 receptor, or SynNotch. In some embodiments, the costimulatory domain comprises 41BB. In some embodiments, the CAR comprises an amino acid sequence that is at least 80% identical to the amino acid sequence of any one of SEQ ID NOs: 2-7. In some embodiments, the CAR comprises the amino acid sequence of any one of SEQ ID NO: 2-7. In some embodiments, the extracellular target binding domain further comprises a signal peptide, optionally wherein the signal peptide comprises a CD27 signal peptide.

Nucleic acids comprising a nucleotide sequence encoding the CAR described herein are also provided. In some embodiments, the nucleotide is operably linked to a promoter. In some embodiments, the promoter is an EF1-alpha promoter.

Vectors comprising the nucleic acids described herein are also provided. In some embodiments, the vector is a retroviral vector, a lentiviral vector or an AAV.

Other aspects of the present disclosure provide engineered immune cells comprising the CAR described herein. In some embodiments, the immune cell is a T-cell, a NK cell, a dendritic cell, a macrophage, a B cell, a neutrophil, an eosinophil, a basophil, a mast cell, a myeloid derived suppressor cell, a mesenchymal stem cell, a precursor thereof, or a combination. In some embodiments, the immune cell is a T-cell. In some embodiments, immune cell is autologous or allogeneic.

Further provided herein are methods comprising administering to a subject the engineered immune cell described herein. In some embodiments, the method is for treating cancer expressing CD70 and comprises administering to a subject in need thereof an effective amount of the engineered immune cell described herein.

In some embodiments, the method of treating a cancer expressing CD70 comprises administering to a subject in need thereof a therapeutically effective amount of the engineered immune cell described herein and an effective amount of an agent that enhances expression of CD70 in the cancer. In some embodiments, the agent results in hypomethylation of CD-70 encoding gene in the cancer. In some embodiments, the agent is azacitidine or decitabine. In some embodiments, the engineered immune cell and the agent are administered simultaneously. In some embodiments, the engineered immune cell and the agent are formulated in a composition. In some embodiments, the agent is azacitidine having a concentration of 10 μM or less in the composition. In some embodiments, the engineered immune cell and the agent are administered sequentially. In some embodiments, the agent is administered before the engineered immune cell is administered. In some embodiments, the method further comprises waiting a period of time between administering the agent and administering the engineered immune cell.

In some embodiments, the subject is human. In some embodiments, the administering is via infusion. In some embodiments, the cancer is a myeloid cancer. In some embodiments, the cancer is acute myeloid leukemia.

The summary above is meant to illustrate, in a non-limiting manner, some of the embodiments, advantages, features, and uses of the technology disclosed herein. Other embodiments, advantages, features, and uses of the technology disclosed herein will be apparent from the Detailed Description, the Drawings, the Examples, and the Claims.

### BRIEF DESCRIPTION OF THE DRAWINGS

The accompanying drawings are not intended to be drawn to scale. In the drawings, each identical or nearly identical component that is illustrated in various FIGs. is represented by a like numeral. For purposes of clarity, not every component may be labeled in every drawing.

In the drawings:

FIGS. 1A-1I. CD70 CAR-T cells proliferated and achieved high transduction efficiencies in healthy human donor T cells and exhibited robust and specific in vitro effector functions in response to CD70+ target cells. (FIG. 1A) CD70 ligand-based CAR construct schematic. (FIG. 1B) CAR construct transduction efficiency assessed by flow cytometry in T cells from 3 healthy donors. (FIG. 1C) CD70 CAR-T cell expansion compared to untransduced T-cells after lentiviral transduction. All differences are nonsignificant (ns) by unpaired t test with Holm-Sidak correction for multiple comparisons. Points represent mean±SEM of T cells from 3 healthy donors. (FIG. 1D) Degranulation of CD70 CAR-T cells after a short term, 4-hour co-culture with Molm13 at a 1:1 ratio. Percentage of BFP+ cells that express CD107a was measured by flow cytometry and is displayed relative to the positive control (phorbol 12-myristate 13-acetate (PMA) and ionomycin). Bars show mean±SEM of CAR-T cells manufactured from three healthy donors. (FIG. 1E) CD70 expression (x-axis) and copy number (y-axis) of characterized AML cell lines using depmap (https://depmap.org/portal/). Cell lines used in this work are highlighted. (FIG. 1F) CD70 expression by flow cytometry of the AML cell lines used with respective isotypes. (FIG. 1G) Levels of cytokines in the supernatants of CD70 CAR-T cells and untransduced T-cells after co-culture for 16 hours with Molm13 at a 1:1 ratio. Cytokines were measured by 12-plex Luminex assay in technical duplicates. Bars show mean±SEM of 3 normal donors. (FIG. 1H) CD70 CAR-T cells generated from 3 health donors were exposed to the indicated cell lines at a 1:1 ratio for 16 hours. Percent of CD70 CAR T (CD3+BFP+) cells expressing CD69 are reported. Bars show mean±SEM. * p<0.05, **** p<0.0001 by 2 way ANOVA with Holm-Sidak multiple comparisons test. (FIG. 1I) Cytotoxicity as assessed in a luciferase-based killing assay for 16 hrs with CD70 CAR T-cells or untransduced T cells (UTD) from three healthy donors against OCI-AML3, Molm13, Monomac1, or THP-1 targets at the indicated effector to target ratios. Data Points indicate ±SEM of triplicates from three healthy donors' T-cells. Experiments repeated with similar results.

FIGS. 2A-2F. CD70 CAR T cells mediated in vivo AML suppression, prolonged survival, and cleared bone marrow blasts. (FIG. 2A) Experimental design: NSG mice were injected with 5×10⁵ Molm13 cells (day −7) and tumor burden was monitored by bioluminescence imaging (BLI) over time. After tumor engraftment and randomization, the mice were treated seven days later (day 0) with a single dose of either 1×10⁶ CAR-T cells or the equivalent number of UTD T cells from the same healthy donor. ((FIG. 2B) Quantification of flux [photons/second] in the experimental groups at the indicated time points. FIG. 2C) BLI of AML xenografts over time in the indicated groups. (FIG. 2D) Kaplan-Meier survival curves of the treatment groups. ** p<0.01 by Log-Rank (Mantel-Cox) test. (FIG. 2E) Quantification of CAR-T cells (CD3+:BFP+) measured in the peripheral blood by flow cytometry. Bars show the median. (FIG. 2F) Percentage of GFP positive cells in the femur at the time of death or euthanasia as assessed by flow cytometry (see FIG. 8 for gating). * p<0.05 by unpaired t-test. Bars show mean±SEM. (FIG. 2G) CD70 expression level was assessed by flow cytometry among bone marrow GFP+ tumor cells. *** p<0.001 by paired t-test. Bars show mean±SEM. Each experiment was repeated with similar results.

FIGS. 3A-3G. Azacitidine treatment, in conjunction with CD70 CAR-T cells, was necessary to eliminate tumor in an aggressive AML model. (FIG. 3A) Experimental design: NSG mice were injected with 5×10⁵ Molm13 cells (day 0) and tumor burden was monitored by BLI biweekly. After tumor engraftment and randomization, mice received IP injections of 2.5 mg/kg/day azacitidine resuspended in PBS or vehicle (PBS alone) starting on day +18 for a duration of 5 days. On day +22 they were treated with either: no intervention, a single dose of CAR-T cells, or the equivalent number of untransduced T-cells (UTD) from the same healthy donor. (FIG. 3B) Quantification of flux [photons/second] in the experimental groups at the indicated time points. (FIG. 3C) Representative BLI of AML xenografts over time in the indicated groups. *** p<0.001 by one-way ANOVA. (FIG. 3D) Kaplan-Meier survival curves of the treatment groups. (FIG. 3E) Quantification of CAR-T cells (CD3+BFP+) measured in the peripheral blood by flow cytometry at the indicated time points. (FIG. 3F) Percentage of GFP positive cells in the femur at the time of death or euthanasia as assessed by flow cytometry. Bars represent the median. ** p<0.01, *** p<0.001, **** p<0.0001 by one-way ANOVA with Tukey's multiple comparisons test. (FIG. 3G) Immunohistochemistry staining for the common human leukocyte antigen (CD45) and human CD3 in the femurs from each of the indicated groups at the time of sacrifice shown at 10× magnification.

FIGS. 4A-4D. Azacitidine (AZA) exposure resulted in increased CD70 expression by Molm13 in vitro and in vivo. (FIG. 4A) OCI-AML3 or Molm13 cells were co-cultured with the indicated concentration of azacitidine for 20 or 43 hours. CD70 surface expression was determined via flow cytometry with gating on live (DAPI−) cells. *** p<0.001, **** p<0.0001 by ANOVA with Holm-Sidak multiple comparisons test. All comparisons between SupT1 concentrations non-significant. (FIG. 4B) Experimental design: NSG mice were injected with 5×10⁵ Molm13 CBG-GFP cells (day 0) and tumor burden was monitored by bioluminescence imaging (BLI) over time to ensure engraftment. (FIG. 4C, FIG. 4D) Bone marrow aspirates were assessed by flow cytometry for the ratio of CD70 in FIG. 4C or the myeloid marker CD33 in FIG. 4D. Median fluorescent intensity (MFI) was calculated relative to an individual isotype. Corresponding flow histograms are shown in FIG. 10 and were gated as in FIG. 8. * p<0.05 by unpaired t-test. Mean is shown ±standard error of the mean (SEM).

FIGS. 5A-5H. CD70 CAR-T cells activated, persisted, and killed in vitro in the presence of therapeutically relevant concentrations of azacitidine. CD70 CAR-T or untransduced (UTD) cells generated from the T-cells of 3 healthy donors were exposed to the indicated levels of azacitidine for 24 hours in the presence of IL-2. Darker bar is CAR, lighter is UTD. Total number of cells (FIG. 5A) and percent viability (FIG. 5B) were assessed. All comparisons to media control nonsignificant by ANOVA and Dunett's multiple comparisons test for FIG. 5A and FIG. 5B. The asterix represents approximate peak bloodstream concentrations of AZA in humans after subcutaneous injection ˜3 μM (35). (FIG. 5C) After a 24-hour incubation in the listed concentrations of AZA, CD70 CAR-T cells were washed and exposed to plate bound CD70 protein overnight. Level of activation was assessed via CD69 expression by flow cytometry. *** p<0.001, **** p<0.0001 by ANOVA and Dunett's multiple comparisons test. Mean is shown ±SEM (FIG. 5D) A 96 well plate was coated with an anti-CD71 antibody followed by inoculation with 125,000 of CD71 natively expressing Molm13-wild type cells per well. After 28 hours of growth, 125,000 CD70 CAR-T cells that had been incubated in the designated concentrations of AZA for 24 hours were washed and added to the plate. Serial measurements of tumor viability (impedance) were taken for 96 hours in a real time cytotoxicity assay. Plots represents CD70 CAR-T cells derived from 3 healthy donors performed in technical duplicate. Mean is shown ±SEM. (FIG. 5E-FIG. 5G) CD70 CAR-T cells generated from the T-cells of 3 healthy individuals were exposed to molm13 targets in a 1:1 ratio for 43 hours at the indicated concentrations of AZA and T-cell subsets were determined via flow cytometry for CD45RA and CCR7 (FIG. 5E), as well as the activation/exhaustion markers PD-1 (FIG. 5F), Tim3 (FIG. 5G), and Lag3 (FIG. 5H). Mean is shown ±SEM. **** p<0.0001 by 2 way ANOVA and Holm-Sidak's multiple comparisons test. Experiment replicated with similar results.

FIGS. 6A-6I. Increased CD70 antigen density resulted in increased CD70 targeted CAR activation and improved tumor control in vivo. (FIG. 6A) CD70 KO Molm13 cells were transduced at various multiplicities of infection (MOI) with truncated CD70 lacking an intracellular signaling domain and under the control of human EF1 alpha promoter. Five populations were selected, and flow sorted for only CD70+ cells, generating five new cell lines, CD70 wild type (CD70WT), CD70 high (CD70high), CD70 high intermediate (CD70high-int), CD70 intermediate (CD70int), CD70 low intermediate (CD70low-int), and CD70 low (CD70low). CD70 expression of the various lines is demonstrated by flow cytometry and number of molecules per cell. (FIG. 6B) CD70 CAR-T cells generated from 3 health donors were exposed to the cells in FIG. 6A at a 1:1 ratio for 16 hours. Percent of CD70 CAR T (CD3+BFP+) cells expressing CD69 are reported. Bars show mean±SEM. * p<0.05, by ANOVA and Dunett's multiple comparisons test. (FIG. 6C) Overnight luciferase based killing assay was performed with the targets in FIG. 6A as well as CD70 KO Molm13 cells. Results using CAR-T cells manufactured from 3 healthy donors are shown. Bars represent ±SEM. No inter-tumor differences were noted other than with CD70− tumor. (FIG. 6D) Levels of various cytokines in the supernatants of untransduced (UTD) T-cells and CD70 CAR T-cells after co-culture for 16 hours with the indicated lines at a 1:1 ratio. Cytokines were measured by 12-plex Luminex assay in technical duplicates. Bars show mean±SEM of 3 normal donors. No differences were noted between tumor groups. (FIG. 6E) In vitro assessment of population doubling rate between the tumors used for in vivo experiment. Bars show mean±SEM. No significant differences by ANOVA and Dunett's multiple comparisons test. (FIG. 6F) 10 NSG mice per group were injected with 5×10⁵ cells from the indicated line (FIG. 11 shows CD70 expression of the lines immediately prior to injection and after several weeks in culture from the time of FIG. 6A) and then treated with a non-curative dose of 1×10⁶ CD70 CAR-T cells from the same donor to identify inter-tumor differences. Images represent BLI at the indicated time points. (FIG. 6G) Summary BLI curves at the indicated time points. Significance determined via unpaired t test and Holm-Sidak method correction for multiple comparisons. ** adjusted p<0.01. (FIG. 6H) Kaplan-Meier survival curves of the treatment groups. *** p <0.001 and **** p<0.0001 by Log-rank (Mantel-Cox) test. (FIG. 61) Day 14 and 21 CAR expansion in the peripheral blood. Bars represent mean±SEM. * p<0.05 by mixed-effects model using Sidak correction for multiple comparisons.

FIG. 7. No statistical difference in CD19 CAR-T cell expansion compared to untransduced T cells (UTD) after lentiviral transduction with CD19 CAR. The CD19-41BB CAR has the same backbone as the CD70-41BB CAR with a CD8 transmembrane domain and CD3zeta intracellular signaling domain. All differences nonsignificant by unpaired t test with Holm-Sidak correction for multiple comparison. Points represent mean±SEM of T cells from 3 healthy donors.

FIGS. 8A-8B. (FIG. 8A) CD70 is expressed in Molm13 WT cells, but not detected in PeCy7 isotypes or CD70 knockout (KO) cells. CD70 expression by flow cytometry of Molm13 wild type, and CD70 CRISPR knockout cell lines compared to isotype control. (FIG. 8B) Cytotoxicity as assessed in a luciferase-based killing assay for 16 hrs with CD70 CAR T-cells or untransduced (UTD) T cells manufactured from three healthy donors against Molm13 CD70 null targets at the indicated effector to target ratios. Bars represent mean±SEM of triplicates from three healthy donors' T-cells. 3:1 Effector (CD70 CAR-T cells):Target (Molm13 WT cells).

FIGS. 9A-9C. Gating strategy for murine femur aspirates where Molm13 cells are labeled with GFP. (FIG. 9A) In vitro flow cytometric appearance of Molm13 CBG-GFP cells by side scatter and GFP (FITC). (FIG. 9B-FIG. 9C) Representative example of marrow aspirate taken from a mouse treated with untransduced T-cells in FIG. 9B or CD70 CAR-T cells in FIG. 9C from FIG. 3. Molm13 cells were identified by GFP expression.

FIGS. 10A-10C. CD70 expression is increased in vivo when mice are treated with azacitidine. CD70 expression in vitro, and in vivo with, or without pretreatment of azacitidine. (FIG. 10A) CD70 expression on in vitro wild type Molm13 cells compared to isotype control. (FIG. 10B, FIG. 10C) Murine negative (TER-119, NK-1.1, Ly-6G, CD11b), GFP (FITC) positive cells were assessed for PeCy7 expression (CD70). An isotype was prepared from the same individual aspirate for each sample. Histograms represent individual (FIG. 10B) azacitidine or (FIG. 10C) vehicle (PBS) treated mice.

FIG. 11. CD70 expression histogram as measured by flow cytometry among the cell lines used for in vivo injection in FIG. 6 prior to murine injection. CD70 expression of the cell lines Molm13 wt, Molm13 CD70−, “8”, and “12” from FIG. 6A was evaluated using flow cytometry immediately prior to murine injection. This data suggests that CD70 expression is effected by truncation of the intracellular signaling domain.

FIGS. 12A-12D. Flow Cytometric Analysis reveals genes that are overexpressed in Primary AML Samples but not Normal Hematopoietic Cells, including CD70. (FIG. 12A) Bulk AML cells. (FIG. 12B) leukemic CD34_CD38−. (FIG. 12C) normal BM CD34+CD38− CD45RA−CD90+ HSCs (blue), CD34+CD38+ progenitors (light blue). (FIG. 12D) CD3+ peripheral blood T-cells (green, freshly purified), brown (activated).

FIG. 13: Tissue expression levels in in different organs shows CD70 is low in concentration or not detectable in healthy tissues assayed.

FIGS. 14A-14D: CD70 CAR T cells mediated in vivo AML suppression leading to prolonged survival and clearance of bone marrow blasts. (FIG. 14A) Experimental design for mouse cancer treatment with CD70 CAR T cells and measurement of bone marrow blasts using flow cytometry. D refers to day. (FIG. 14B) CD70 CAR T cell treatment reduces tumor expansion. (FIG. 14C) Treatment with CD70 CAR T cells increases survival over no treatment controls (UTD and Tumor Only). Increased number of CD70 CAR T cells (1×10⁶ to 2×10⁶) resulted in increased survival. (FIG. 14D) Days after CAR injection vs. number of CD3+BFP+ cells per μL of blood for two different CD70 doses (1×10⁶ and 2×10⁶).

FIG. 15. 10 μM AZA causes bulk increase in CD70 expression. Quantified using flow cytometry.

FIG. 16. Proposed mechanism of synergy between CD70 CAR-T cells and azacitidine. Upper portion: after saline pre-treatment, CD70 CAR-T administration does not result in tumor control of leukemia engrafted mice. Lower portion: pretreatment with azacitidine results in increased CD70 tumor expression, CAR-T expansion, trafficking to the bone marrow, and tumor clearance.

### DETAILED DESCRIPTION OF CERTAIN EMBODIMENTS

While chimeric antigen receptor (CAR) T cell therapy has resulted in dramatic responses in lymphoid malignancies, targeting myeloid diseases remains a substantial challenge in part due to the lack of tumor-specific antigens, and potential for on-target off-tumor toxicities with lineage antigens. Furthermore, efficacy of existing CAR therapies can be compromised via target antigen loss or downregulation.

The present disclosure, in some aspects, provide CAR-T cells targeting the tumor necrosis alpha family member, CD70 and the use of the CAR-T cells for the treatment of hematologic malignancies (e.g., acute myeloid leukemia (AML)). CD70 is consistently expressed on myeloid blasts and leukemic stem cells but is highly restricted expression in healthy human tissues. As demonstrated herein, CD70-targeting CAR-T cells achieved antigen-specific activation, cytokine production, and cytotoxic activity in models of leukemia in vitro and in vivo. It was further demonstrated herein that, surprisingly, CD70-targeting CAR-T cells were synergistic in vivo in combination with the anti-leukemic hypomethylating drug azacitidine, and the potency of the CAR-T cells was augmented by azacitidine via increasing CD70 expression in the cancer cells.

Some aspects of the present disclosure provide chimeric antigen receptors (CARs) comprising: (i) an extracellular target binding domain comprising a polypeptide that binds CD70; (ii) a transmembrane domain; and (iii) an intracellular signaling domain.

A “chimeric antigen receptor (CAR)” refers to a receptor protein that has been engineered to perform both antigen-binding and cell activating functions. In some embodiments, a CAR comprises a plurality of linked domains having distinct functions. CAR domains include those with antigen-binding functions, those with structural functions, and those with signaling functions. In some embodiments, a CAR comprises at least an extracellular ligand domain, a transmembrane domain and a cytoplasmic signaling domain (also referred to herein as “an intracellular signaling domain”) comprising a functional signaling domain derived from a stimulatory molecule as defined below. In some embodiments, the CAR comprises an optional leader sequence (also referred to as “signal peptide”), an extracellular antigen binding domain, a hinge, a transmembrane domain, and an intracellular stimulatory domain. In some embodiments, the domains in the CAR are in the same polypeptide chain, e.g., comprise a chimeric fusion protein. In some embodiments, the domains in the CAR are not contiguous with each other.

In some embodiments, the CAR described herein comprises an extracellular target binding domain comprising a polypeptide that binds Cluster of Differentiation 70 (CD70). “CD70” refers to a polypeptide that is encoded by the human CD70 gene (NCBI Gene ID: 970). As described herein, expression of CD70 is highly restricted in normal human (non-cancer) tissues. However, CD70 is expressed in numerous cancers, for example, bladder cancer, breast invasive carcinoma, cervical cancer, cholangiocarcinoma, colorectal cancer, diffuse large B-cell lymphoma (DLBC), Esophagus, glioblastoma (GBM), head and neck cancer, low-grade gliomas (LGG), liver cancer, lung adeno cancer, melanoma, mesothelioma, ovarian cancer, pancreatic cancer, prostate cancer, sarcoma, stomach cancer, testicular germ cell cancer, thymoma, thyroid cancer, uterine cancer, uveal melanoma, clear cell renal cell carcinoma (ccRCC), chromophobe renal cell carcinoma, papillary renal cell carcinoma (pRCC), acute myeloid leukemia, and adenoid cystic carcinoma (ACC) (Pan-Cancer Atlas 2018). CD70 is a cytokine that contains a cytoplasmic, transmembrane, and extracellular domains. The extracellular domain of CD70 is a ligand for CD27.

In some embodiments, the polypeptide that binds CD70 comprises a CD70-binding domain of Cluster of Differentiation 27 (CD27) also called the CD27 antigen. “CD27” refers to a polypeptide that is encoded by the human CD27 gene (NCBI GENE ID: 939, Uniprot ID: P26842). An example of the CD27 amino acid sequence is provided below.

(SEQ ID NO: 8)
MARPHPWWLCVLGTLVGLSATPAPKSCPERHYWAQGKLCCQMCEPGTFL
VKDCDQHRKAAQCDPCIPGVSFSPDHHTRPHCESCRHCNSGLLVRNCTI
TANAECACRNGWQCRDKECTECDPLPNPSLTARSSQALSPHPQPTHLPY
VSEMLEARTAGHMQTLADFRQLPARTLSTHWPPQRSLCSSDFIRILVIF
SGMFLVFTLAGALFLHQRRKYRSNKGESPVEPAEPCHYSCPREEEGSTI
PIQEDYRKPEPACSP

The CD27 protein has extracellular, transmembrane, and cytoplasmic domains. In some embodiments, the CD70 binding domain is located within the extracellular signaling domain of CD27. In some embodiments, the extracellular region contains multiple cysteine-rich domains (CRD): CDR1, CDR2, and CDR3. In some embodiments, the CD70 binding domain is located within the CRD2 domain.

In some embodiments, the CD70-binding domain in CD27 comprises a peptide comprising the amino acid sequence of TRPHCESCRHCN (SEQ ID NO: 9) that is located in the extracellular domain of CD27. In some embodiments, the extracellular targeting binding domain of the CAR described herein comprises a polypeptide comprising an amino acid sequence that is at least 70% identical (e.g., at least 70%, at least 80%, at least 90%, or at least 95% identical) to the amino acid sequence of SEQ ID NO: 9. In some embodiments, the extracellular targeting binding domain of the CAR described herein comprises the amino acid sequence of SEQ ID NO: 9.

In some embodiments, the extracellular targeting binding domain of the CAR described herein comprises a polypeptide comprising the extracellular domain of CD27. In some embodiments, the extracellular targeting binding domain of the CAR described herein comprises a polypeptide comprising an amino acid sequence that is at least 70% identical (e.g., at least 70%, at least 80%, at least 90%, or at least 95% identical) to the amino acid sequence of SEQ ID NO: 1. In some embodiments, the extracellular targeting binding domain of the CAR described herein comprises a polypeptide comprising the amino acid sequence of SEQ ID NO: 1.

In some embodiments, the polypeptide that binds CD70 in the extracellular targeting binding domain of the CAR described herein comprises an anti-CD70 antibody. The term “antibody,” used herein encompasses antibodies of different formats and antibody fragments. In some embodiments, antibody includes but is not limited to a monoclonal antibody, a polyclonal antibody, a recombinant antibody, a human antibody, a humanized antibody, and a functional fragment thereof, including but not limited to a single-chain variable fragment (scFV), a single-domain antibody such as a heavy chain variable domain (VH), a light chain variable domain (VL) and a variable domain (VHH) of camelid derived nanobody, and to an alternative scaffold known in the art to function as antigen binding domain, such as a recombinant fibronectin domain, and the like. In some embodiments, it is beneficial for the antigen binding domain to be derived from the same species in which the CAR will ultimately be used in. For example, for use in humans, it may be beneficial for the antigen binding domain of the CAR to comprise human or humanized residues for the antigen binding domain of an antibody or antibody fragment. In some embodiments, the polypeptide that binds CD70 in the extracellular targeting binding domain of the CAR described herein comprises a scFv that binds to CD70.

In some embodiments, the antibody is a human antibody or an antibody fragment. In some embodiments, the antibody a humanized antibody or an antibody fragment. A humanized antibody can be produced using a variety of techniques known in the art, including but not limited to, CDR-grafting (see, e.g., European Patent No. EP 239,400; International Publication No. WO 91/09967; and U.S. Pat. Nos. 5,225,539, 5,530,101, and 5,585,089, each of which is incorporated herein in its entirety by reference), veneering or resurfacing (see, e.g., European Patent Nos. EP 592,106 and EP 519,596; Padlan, 1991, Molecular Immunology, 28(4/5):489-498; Studnicka et al., 1994, Protein Engineering, 7(6):805-814; and Roguska et al., 1994, PNAS, 91:969-973, each of which is incorporated herein by its entirety by reference), chain shuffling (see, e.g., U.S. Pat. No. 5,565,332, which is incorporated herein in its entirety by reference), and techniques disclosed in, e.g., U.S. Patent Application Publication No. US2005/0042664, U.S. Patent Application Publication No. US2005/0048617, U.S. Pat. Nos. 6,407,213, 5,766,886, International Publication No. WO 9317105, Tan et al., J. Immunol., 169:1119-25 (2002), Caldas et al., Protein Eng., 13(5):353-60 (2000), Morea et al., Methods, 20(3):267-79 (2000), Baca et al., J. Biol. Chem., 272(16):10678-84 (1997), Roguska et al., Protein Eng., 9(10):895-904 (1996), Couto et al., Cancer Res., 55 (23 Supp):5973s-5977s (1995), Couto et al., Cancer Res., 55(8):1717-22 (1995), Sandhu J S, Gene, 150(2):409-10 (1994), and Pedersen et al., J. Mol. Biol., 235(3):959-73 (1994), each of which is incorporated herein in its entirety by reference. Often, framework residues in the framework regions will be substituted with the corresponding residue from the CDR donor antibody to alter, for example improve, antigen binding. These framework substitutions are identified by methods well-known in the art, e.g., by modeling of the interactions of the CDR and framework residues to identify framework residues important for antigen binding and sequence comparison to identify unusual framework residues at particular positions. (See, e.g., Queen et al., U.S. Pat. No. 5,585,089; and Riechmann et al., 1988, Nature, 332:323, which are incorporated herein by reference in their entireties.)

A humanized antibody or antibody fragment has one or more amino acid residues remaining in it from a source which is non-human. These non-human amino acid residues are often referred to as “import” residues, which are typically taken from an “import” variable domain. As provided herein, humanized antibodies or antibody fragments comprise one or more CDRs from non-human immunoglobulin molecules and framework regions wherein the amino acid residues comprising the framework are derived completely or mostly from human germline. Multiple techniques for humanization of antibodies or antibody fragments are well-known in the art and can essentially be performed following the method of Winter and co-workers (Jones et al., Nature, 321:522-525 (1986); Riechmann et al., Nature, 332:323-327 (1988); Verhoeyen et al., Science, 239:1534-1536 (1988)), by substituting rodent CDRs or CDR sequences for the corresponding sequences of a human antibody, i.e., CDR-grafting (EP 239,400; PCT Publication No. WO 91/09967; and U.S. Pat. Nos. 4,816,567; 6,331,415; 5,225,539; 5,530,101; 5,585,089; 6,548,640, the contents of which are incorporated herein by reference herein in their entirety). In such humanized antibodies and antibody fragments, substantially less than an intact human variable domain has been substituted by the corresponding sequence from a non-human species. Humanized antibodies are often human antibodies in which some CDR residues and possibly some framework (FR) residues are substituted by residues from analogous sites in rodent antibodies. Humanization of antibodies and antibody fragments can also be achieved by veneering or resurfacing (EP 592,106; EP 519,596; Padlan, 1991, Molecular Immunology, 28(4/5):489-498; Studnicka et al., Protein Engineering, 7(6):805-814 (1994); and Roguska et al., PNAS, 91:969-973 (1994)) or chain shuffling (U.S. Pat. No. 5,565,332), the contents of which are incorporated herein by reference herein in their entirety.

In some embodiments, the antibody is derived from a display library. A display library is a collection of entities; each entity includes an accessible polypeptide component and a recoverable component that encodes or identifies the polypeptide component. The polypeptide component is varied so that different amino acid sequences are represented. The polypeptide component can be of any length, e.g., from three amino acids to over 300 amino acids. A display library entity can include more than one polypeptide component, for example, the two polypeptide chains of a Fab. In one exemplary embodiment, a display library can be used to identify an antigen binding domain. In a selection, the polypeptide component of each member of the library is probed with the antigen, or a fragment there, and if the polypeptide component binds to the antigen, the display library member is identified, typically by retention on a support.

Retained display library members are recovered from the support and analyzed. The analysis can include amplification and a subsequent selection under similar or dissimilar conditions. For example, positive and negative selections can be alternated. The analysis can also include determining the amino acid sequence of the polypeptide component and purification of the polypeptide component for detailed characterization.

A variety of formats can be used for display libraries. Examples include the phage display. In phage display, the protein component is typically covalently linked to a bacteriophage coat protein. The linkage results from translation of a nucleic acid encoding the protein component fused to the coat protein. The linkage can include a flexible peptide linker, a protease site, or an amino acid incorporated as a result of suppression of a stop codon. Phage display is described, for example, in U.S. Pat. No. 5,223,409; WO 92/18619; WO 91/17271; WO 92/20791; WO 92/15679; WO 93/01288; WO 92/01047; WO 92/09690; WO 90/02809. Bacteriophage displaying the protein component can be grown and harvested using standard phage preparatory methods, e.g. PEG precipitation from growth media. After selection of individual display phages, the nucleic acid encoding the selected protein components can be isolated from cells infected with the selected phages or from the phage themselves, after amplification. Individual colonies or plaques can be picked, the nucleic acid isolated and sequenced. Other display formats include cell based display (see, e.g., WO 03/029456), protein-nucleic acid fusions (see, e.g., U.S. Pat. No. 6,207,446), ribosome display, and E. coli periplasmic display.

The transmembrane domain of the CARs described herein may be derived either from a natural or from a recombinant source. Where the source is natural, the domain may be derived from any membrane-bound or transmembrane protein. In one aspect the transmembrane domain is capable of signaling to the intracellular domain(s) whenever the CAR has bound to a target. A transmembrane domain of particular use in this invention may include at least the transmembrane region(s) of e.g., the alpha, beta or zeta chain of the T-cell receptor, CD28, CD3 epsilon, CD45, CD4, CD5, CD8 (e.g., CD8 alpha, CD8 beta), CD9, CD16, CD22, CD27, CD33, CD37, CD64, CD80, CD86, CD134, CD137, CD154. In some embodiments, a transmembrane domain may include at least the transmembrane region(s) of, e.g., KIRDS2, OX40, CD2, CD27, LFA-1 (CD11a, CD18), ICOS (CD278), 4-1BB (CD137), GITR, CD40, BAFFR, HVEM (LIGHTR), SLAMF7, NKp80 (KLRF1), NKp44, NKp30, NKp46, CD160, CD19, IL2R beta, IL2R gamma, IL7R a, ITGA1, VLA1, CD49a, ITGA4, IA4, CD49D, ITGA6, VLA-6, CD49f, ITGAD, CD11d, ITGAE, CD103, rfGAL, CD11a, LFA-1, ITGAM, CD11b, ITGAX, CD11c, ITGB1, CD29, ITGB2, CD18, LFA-1, ITGB7, TNFR2, DNAM1 (CD226), SLAMF4 (CD244, 2B4), CD84, CD96 (Tactile), CEACAM1, CRT AM, Ly9 (CD229), CD160 (BY55), PSGL1, CD100 (SEMA4D), SLAMF6 (NTB-A, Ly108), SLAM (SLAMF1, CD150, IPO-3), BLAME (SLAMF8), SELPLG (CD162), LTBR, PAG/Cbp, NKG2D, NKG2C, and CD19. In some embodiments, the transmembrane domain is a CD28 transmembrane domain or CD8 transmembrane domain. In some embodiments, transmembrane domain is the transmembrane domain of CD27. In some embodiments, the transmembrane domain of CD27 comprises an amino acid sequence of ILVIFSGMFLVFTLAGALFL (SEQ ID NO: 10).

In some embodiments, the transmembrane domain can be attached to the extracellular region of the CAR, e.g., the ligand domain of the CAR, via a hinge, e.g., a hinge from a human protein. For example, in one embodiment, the hinge can be a human Ig (immunoglobulin) hinge, e.g., an IgG4 hinge, or a CD8a hinge.

In some embodiments, the cytoplasmic domain or region of the CAR described herein includes one or more intracellular signaling domains. An intracellular signaling domain is capable of activation of at least one of the normal effector functions of the immune cell in which the CAR has been introduced. Examples of intracellular signaling domains for use in the CAR described herein include the cytoplasmic sequences of the T cell receptor (TCR) and co-receptors that act in concert to initiate signal transduction following antigen receptor engagement, as well as any derivative or variant of these sequences and any recombinant sequence that has the same functional capability.

T cell activation can be said to be mediated by two distinct classes of cytoplasmic signaling sequences: those that initiate antigen-dependent primary activation through the TCR (primary intracellular signaling domains) and those that act in an antigen-independent manner to provide a secondary or costimulatory signal (secondary cytoplasmic domain, e.g., a costimulatory domain).

An “intracellular signaling domain,” as the term is used herein, refers to an intracellular portion of a molecule. The intracellular signaling domain can generate a signal that promotes an immune effector function of the CAR containing cell, e.g., a CAR T cell or CAR-expressing NK cell. Examples of immune effector function, e.g., in a CAR T cell or CAR-expressing NK cell, include cytolytic activity and helper activity, including the secretion of cytokines. In embodiments, the intracellular signal domain transduces the effector function signal and directs the cell to perform a specialized function. While the entire intracellular signaling domain can be employed, in many cases it is not necessary to use the entire chain. To the extent that a truncated portion of the intracellular signaling domain is used, such truncated portion may be used in place of the intact chain as long as it transduces the effector function signal. The term intracellular signaling domain is thus meant to include any truncated portion of the intracellular signaling domain sufficient to transduce the effector function signal.

In some embodiments, the one or more intracellular signaling domains comprise a primary intracellular signaling domain. Exemplary primary intracellular signaling domains include those derived from the molecules responsible for primary stimulation, or antigen dependent simulation. In some embodiments, a primary intracellular signaling domain comprises a signaling motif which is known as an immunoreceptor tyrosine-based activation motif or ITAM. Examples of ITAM containing primary cytoplasmic signaling sequences include, but are not limited to, those derived from CD3 zeta, FcR gamma, FcR beta, CD3 gamma, CD3 delta, CD3 epsilon, CD3 theta, CD3 eta, CD5, CD22, CD79a, CD79b, CD278 (“ICOS”), FceRI, CD66d, DAP10, and DAP12. In some embodiments, the intracellular signaling domain of the CAR comprises a CD3-zeta (CD3t) signaling domain. In some embodiments, the CD3-zeta (CD3) signaling domain comprises the amino acid sequence of: RVKFSRSADAPAYQQGQNQLYNELNLGRREEYDVLDKRRGRDPEMGGKPRRKNPQE GLYNELQKDKMAEAYSEIGMKGERRRGKGHDGLYQGLSTATKDTYDALHMQALPP R (SEQ ID NO: 11). In some embodiments, the CD3-zeta (CD3t) signaling domain of the CAR described herein comprises an amino acid sequence that is at least 70% identical (e.g., at least 70%, at least 80%, at least 90%, or at least 95% identical) to the amino acid sequence of SEQ ID NO: 11.

In some embodiments, the one or more intracellular signaling domain comprise a costimulatory intracellular domain. A costimulatory intracellular signaling domain refers to the intracellular portion of a costimulatory molecule. The intracellular signaling domain can comprise the entire intracellular portion, or the entire native intracellular signaling domain, of the molecule from which it is derived, or a functional fragment thereof. Exemplary costimulatory intracellular signaling domains include those derived from molecules responsible for costimulatory signals (e.g., antigen independent stimulation), and those derived from cytokine receptors. In some embodiments, the one or more intracellular signaling domains comprise a primary intracellular signaling domain, and a costimulatory intracellular signaling domain from one or more co-stimulatory proteins or cytokine receptors.

The term “costimulatory molecule” refers to the cognate binding partner on a T cell that specifically binds with a costimulatory ligand, thereby mediating a costimulatory response by the T cell, such as, but not limited to, proliferation. Costimulatory molecules are cell surface molecules other than antigen receptors or their ligands that are required for an efficient immune response. Examples of such molecules include a MHC class I molecule, TNF receptor proteins, Immunoglobulin-like proteins, cytokine receptors, integrins, signaling lymphocytic activation molecules (SLAM proteins), activating NK cell receptors, BTLA, a Toll ligand receptor, OX40, CD2, CD7, CD27, CD28, CD30, CD40, CDS, ICAM-1, LFA-1 (CD1 1a/CD18), 4-1BB (CD137), B7-H3, CDS, ICAM-1, ICOS (CD278), GITR, BAFFR, LIGHT, HVEM (LIGHTR), KIRDS2, SLAMF7, NKp80 (KLRF1), NKp44, NKp30, NKp46, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, CD11c, ITGB1, CD29, ITGB2, CD18, LFA-1, ITGB7, NKG2D, NKG2C, TNFR2, TRANCE/RANKL, DNAM1 (CD226), SLAMF4 (CD244, 2B4), CD84, CD96 (Tactile), CEACAM1, CRT AM, Ly9 (CD229), CD160 (BY55), PSGL1, CDIOO (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, CD19a, and a ligand that specifically binds with CD83. For example, CD27 co-stimulation has been demonstrated to enhance expansion, effector function, and survival of human CART cells in vitro and augments human T cell persistence and antitumor activity in vivo (Song et al. Blood. 2012; 119(3):696-706). In some embodiments, the co-stimulatory domain of the CARs described herein comprises on or more signaling domains from one or more co-stimulatory protein or cytokine receptor selected from CD28, 4-1BB, 2B4, KIR, CD27, OX40, ICOS, MYD88, IL2 receptor, and SynNotch. In some embodiments, the co-stimulatory domain of the CARs described herein comprises a 4-1BB costimulatory signaling domain. In some embodiments, the 4-1BB co-stimulatory signaling domain comprises the amino acid sequence of: KRGRKKLLYIFKQPFMRPVQTTQEEDGCSCRFPEEEEGGCEL (SEQ ID NO: 12). In some embodiments, the 4-1BB co-stimulatory signaling domain of the CAR described herein comprises an amino acid sequence that is at least 70% identical (e.g., at least 70%, at least 80%, at least 90%, or at least 95% identical) to the amino acid sequence of SEQ ID NO: 12.

In some embodiments, the intracellular signaling domain of the CAR described herein comprise the primary signaling domain, e.g., an ITAM containing domain such as a CD3-zeta signaling domain, by itself or combined with a costimulatory signaling domain (e.g., a co-stimulating domain from one or more co-stimulatory protein or cytokine receptor selected from CD28, 4-1BB, 2B4, KIR, CD27, OX40, ICOS, MYD88, IL2 receptor, and SynNotch). In some embodiments, the intracellular signaling domain of the CAR described herein comprise a CD3-zeta (CD3ζ) signaling domain and a 4-1BB costimulatory signaling domain.

In some embodiments, different linker sequences may be used between the different domains of the CAR, e.g., a (GGGS)n linker, wherein n is 1-20 (e.g., 1, 2, 3, 4, 5, 6, 7, 8, 9, 10, 11, 12, 13, 14, 15, 16, 17, 18, 19, or 20). In some embodiments, the linker is (GGGS)₇. In some embodiments, the CAR comprises additional sequences from CD27, e.g., the stalk and hinge region of CD27, between the extracellular target binding domain and the transmembrane region. In some embodiments, the stalk and hinge region of CD27 comprises the amino acid sequence of: PLPNPSLTARSSQALSPHPQPTHLPYVSEMLEARTAGHMQTLADFRQLPARTLSTHWP PQRSLCSSDFIR (SEQ ID NO: 13). In some embodiments, the CAR does not comprise additional sequences from CD27, e.g., the stalk and hinge region of the between the extracellular target binding domain and the transmembrane region.

In some embodiments, the CAR described herein comprises an amino acid sequence that is at least 70% identical (e.g., at least 70%, at least 80%, at least 90%, or at least 95% identical) to the amino acid sequence of any one of SEQ ID NOs: 2-7. In some embodiments, the CAR described herein comprises the amino acid sequence of any one of SEQ ID NOs: 2-7.

In some embodiments, the CARs described herein further comprises a leader sequence (also referred herein to as a signal peptide) at the amino-terminus (N-terminus) of the antigen binding domain. In some embodiments, the CAR further comprises a leader sequence at the N-terminus of the antigen binding domain, wherein the leader sequence is optionally cleaved from the antigen binding domain (e.g., a scFv) during cellular processing and localization of the CAR to the cellular membrane. In some embodiments, the leader sequence is a CD27 signal peptide (e.g., a peptide having the amino acid sequence of: MARPHPWWLCVLGTLVGLS (SEQ ID NO: 14)) In some embodiments, the leader sequence is an interleukin 2 signal peptide or a CD8 leader sequence. In some embodiments, the leader sequence comprises an amino acid sequence of: MALPVTALLLPLALLLHAARP (SEQ ID NO: 15).

In some embodiments, the CARs described herein further comprises additional amino acid sequences (e.g., between the extracellular target binding domain and the leader sequence. In some embodiments, the additional sequence is an affinity tag (e.g., a Myc tag, EQKLISEEDL (SEQ ID NO: 16).

In some aspects, the disclosure provides nucleic acid molecules (e.g., vectors) for expressing CARs in cells, e.g., T cells. In some embodiments, the nucleic acid molecule comprises a nucleotide sequence encoding the CAR described herein. The nucleic acid sequences coding for the desired molecules can be obtained using recombinant methods known in the art, such as, for example by screening libraries from cells expressing the gene, by deriving the gene from a vector known to include the same, or by isolating directly from cells and tissues containing the same, using standard techniques. Recombinant DNA and molecular cloning techniques used here are well known in the art and are described, for example, by Sambrook, J., Fritsch, E. F. and Maniatis, T. MOLECULAR CLONING: A LABORATORY MANUAL, 2nd ed.; Cold Spring Harbor Laboratory: Cold Spring Harbor, N.Y., 1989; and by Silhavy, T. J., Bennan, M. L. and Enquist, L. W. EXPERIMENTS WITH GENE FUSIONS; Cold Spring Harbor Laboratory: Cold Spring Harbor, N.Y., 1984; and by Ausubel, F. M. et al., IN CURRENT PROTOCOLS IN MOLECULAR BIOLOGY, published by Greene Publishing and Wiley-Interscience, 1987; (the entirety of each of which is hereby incorporated herein by reference). Alternatively, the gene of interest can be produced synthetically, rather than cloned.

In some embodiments, the desired CAR can be expressed in the cells by way of transposons. In some embodiments, expression of natural or synthetic nucleic acids CARs is typically achieved by operably linking a nucleic acid encoding the CAR to a promoter, and incorporating the construct into an expression vector. The vectors can be suitable for replication and integration into eukaryotes. Typical cloning vectors contain transcription and translation terminators, initiation sequences, and promoters useful for regulation of the expression of the desired nucleic acid sequence. The expression constructs of the disclosure may also be used for nucleic acid immunization and gene therapy, using standard gene delivery protocols. Methods for gene delivery are known in the art. See, e.g., U.S. Pat. Nos. 5,399,346, 5,580,859, 5,589,466, incorporated by reference herein in their entireties.

Additional promoter elements, e.g., enhancers, regulate the frequency of transcriptional initiation. Typically, these are located in the region 30-110 bp upstream of the start site, although a number of promoters have recently been shown to contain functional elements downstream of the start site as well. The spacing between promoter elements frequently is flexible, so that promoter function is preserved when elements are inverted or moved relative to one another. In the thymidine kinase (tk) promoter, the spacing between promoter elements can be increased to 50 bp apart before activity begins to decline. Depending on the promoter, it appears that individual elements can function either cooperatively or independently to activate transcription.

One example of a suitable promoter is the immediate early cytomegalovirus (CMV) promoter sequence. This promoter sequence is a strong constitutive promoter sequence capable of driving high levels of expression of any polynucleotide sequence operatively linked thereto. Another example of a suitable promoter is Elongation Factor-1a (EF-1a). However, other constitutive promoter sequences may also be used, including, but not limited to the simian virus 40 (SV40) early promoter, mouse mammary tumor virus (MMTV), human immunodeficiency virus (HIV) long terminal repeat (LTR) promoter, MoMuLV promoter, an avian leukemia virus promoter, an Epstein-Barr virus immediate early promoter, a Rous sarcoma virus promoter, as well as human gene promoters such as, but not limited to, the actin promoter, the myosin promoter, the hemoglobin promoter, and the creatine kinase promoter. Further, the disclosure is not limited to the use of constitutive promoters. Inducible promoters are also contemplated as part of the disclosure. The use of an inducible promoter provides a molecular switch capable of turning on expression of the polynucleotide sequence which it is operatively linked when such expression is desired, or turning off the expression when expression is not desired. Examples of inducible promoters include, but are not limited to a metallothionine promoter, a glucocorticoid promoter, a progesterone promoter, and a tetracycline promoter. In some embodiments, the promoter is an EF-1a promoter.

In some embodiments, the nucleic acid comprising a nucleotide sequence encoding the CAR described herein is a vector. The nucleic acid can be cloned into a number of types of vectors. For example, the nucleic acid can be cloned into a vector including, but not limited to a plasmid, a phagemid, a phage derivative, an animal virus, and a cosmid. Vectors of particular interest include expression vectors, replication vectors, probe generation vectors, and sequencing vectors.

Further, the expression vector may be provided to a cell in the form of a viral vector. Viral vector technology is well known in the art and is described, for example, in Sambrook et al. (2001, Molecular Cloning: A Laboratory Manual, Cold Spring Harbor Laboratory, New York), and in other virology and molecular biology manuals. Viruses, which are useful as vectors include, but are not limited to, retroviruses, adenoviruses, adeno-associated viruses, herpes viruses, and lentiviruses. In general, a suitable vector contains an origin of replication functional in at least one organism, a promoter sequence, convenient restriction endonuclease sites, and one or more selectable markers (e.g., WO 01/96584; WO 01/29058; and U.S. Pat. No. 6,326,193).

A number of viral based systems have been developed for gene transfer into mammalian cells. For example, retroviruses provide a convenient platform for gene delivery systems. A selected gene can be inserted into a vector and packaged in retroviral particles using techniques known in the art. The recombinant virus can then be isolated and delivered to cells of the subject either in vivo or ex vivo. In some embodiments, retrovirus vectors are used. In some embodiments, lentivirus vectors are used. In some embodiments, adeno-associated virus (AAV) vectors can also be used.

Vectors derived from retroviruses such as the lentivirus are suitable tools to achieve long-term gene transfer since they allow long-term, stable integration of a transgene and its propagation in daughter cells. Lentiviral vectors have the added advantage over vectors derived from onco-retroviruses such as murine leukemia viruses in that they can transduce non-proliferating cells, such as hepatocytes. They also have the added advantage of low immunogenicity. A “lentivirus” as used herein refers to a genus of the Retroviridae family. Lentiviruses are unique among the retroviruses in being able to infect non-dividing cells; they can deliver a significant amount of genetic information into the DNA of the host cell, so they are one of the most efficient methods of a gene delivery vector. HIV, SIV, and FIV are all examples of lentiviruses. Vectors derived from lentiviruses offer the means to achieve significant levels of gene transfer in vivo.

Any methods known in the art for delivering nucleic acids or proteins into a cell may be used, e.g., transfection, transformation, transduction, or electroporation. The term “transfected” or “transformed” or “transduced” as used herein refers to a process by which exogenous nucleic acid is transferred or introduced into the host cell. A “transfected” or “transformed” or “transduced” cell is one which has been transfected, transformed or transduced with exogenous nucleic acid. The cell includes the primary subject cell and its progeny.

Other aspects of the present disclosure provide engineered immune cell comprising the CAR or the nucleic acid encoding the CAR described herein. In some embodiments, the immune cell is a mammalian immune cell. In some embodiments, the immune cell is a human immune cell. An “immune cell” can be a T-cell, an NK cell, a dendritic cell, a macrophage, a B cell, a neutrophil, an eosinophil, a basophil, a mast cell, a myeloid-derived suppressor cell, a mesenchymal stem cell, or combinations thereof, or any precursor, derivative, or progenitor cells thereof. In some embodiments, the immune cell is a T cell. In some embodiments, the immune cell is a human T cell.

Immune cells (e.g., T cells) can be obtained from a number of sources, including peripheral blood mononuclear cells, bone marrow, lymph node tissue, cord blood, thymus tissue, tissue from a site of infection, ascites, pleural effusion, spleen tissue, and tumors. The immune cells (e.g., T cells) may also be generated from induced pluripotent stem cells or hematopoietic stem cells or progenitor cells. In some embodiments, any number of immune cell lines, including but not limited to T cell lines, including, for example, Hep-2, Jurkat, and Raji cell lines, available in the art, may be used. In some embodiments, immune cells (e.g., T cells) can be obtained from a unit of blood collected from a subject using any number of techniques known to the skilled artisan, such as Ficoll™ separation. In some embodiments, cells from the circulating blood of an individual are obtained by apheresis. The apheresis product typically contains lymphocytes, including T cells, monocytes, granulocytes, B cells, NK cells, other nucleated white blood cells, red blood cells, and platelets. In some embodiments, the cells collected by apheresis may be washed to remove the plasma fraction and to place the cells in an appropriate buffer or media for subsequent processing steps. In some embodiments, the cells are washed with phosphate buffered saline (PBS). In some embodiments, the wash solution lacks calcium and may lack magnesium or may lack many if not all divalent cations. Again, surprisingly, initial activation steps in the absence of calcium lead to magnified activation. As those of ordinary skill in the art would readily appreciate a washing step may be accomplished by methods known to those in the art, such as by using a semi-automated “flow-through” centrifuge (for example, the Cobe 2991 cell processor, the Baxter CytoMate, or the Haemonetics Cell Saver 5) according to the manufacturer's instructions. After washing, the cells may be resuspended in a variety of biocompatible buffers, such as, for example, Ca²⁺-free, Mg²⁺-free PBS, PlasmaLyte A, or other saline solution with or without buffer. Alternatively, the undesirable components of the apheresis sample may be removed and the cells directly resuspended in culture media.

In some embodiments, immune cells (e.g., T cells) are isolated from peripheral blood lymphocytes by lysing the red blood cells and depleting the monocytes, for example, by centrifugation through a PERCOLL™ gradient or by counterflow centrifugal elutriation. A specific subpopulation of T cells, such as CD3⁺, CD28⁺, CD4⁺, CD8⁺, CD45RA⁺, and CD45RO⁺T cells, can be further isolated by positive or negative selection techniques.

Enrichment of a T cell population by negative selection can be accomplished with a combination of antibodies directed to surface markers unique to the negatively selected cells. One method is cell sorting and/or selection via negative magnetic immunoadherence or flow cytometry that uses a cocktail of monoclonal antibodies directed to cell surface markers present on the cells negatively selected. For example, to enrich for CD4⁺ cells by negative selection, a monoclonal antibody cocktail typically includes antibodies to CD14, CD20, CD1 lb, CD16, HLA-DR, and CD8. In certain embodiments, it may be desirable to enrich for or positively select for regulatory T cells which typically express CD4⁺, CD25⁺, CD62L^{hi}, GITR⁺, and FoxP3⁺. Alternatively, in some embodiments, T regulatory cells are depleted by anti-C25 conjugated beads or other similar method of selection.

The engineered immune cells (e.g., T cells) may be autologous. Being “autologous” means the immune cells are obtained from a subject, engineered to express a CAR described herein, and administered to the same subject. Administration of autologous cells to a subject may result in reduced rejection of the immune cells as compared to administration of non-autologous cells. Alternatively, the engineered immune cells (e.g., T cells) can be allogeneic cells. Being “allogeneic” the cells are obtained from a first subject, modified to express the CAR described herein and administered to a second subject that is different from the first subject but of the same species. For example, allogeneic immune cells may be derived from a human donor and administered to a human recipient who is different from the donor.

Other aspects of the present disclosure provide compositions comprising any one of the engineered immune cells (e.g., CD70-targeting CAR-T cells) described herein. In some embodiments, the composition comprising the engineered immune cells (e.g., CD70-targeting CAR-T cells) further comprises an agent that enhances CD70 expression in cancer cells. In some embodiments, the agent results in hypomethylation of CD-70 encoding gene in the cancer. In some embodiments, the agent is azacitidine or decitabine. In some embodiments, the composition comprises the engineered immune cells (e.g., CD70-targeting CAR-T cells) and azacitidine. In some embodiments, the composition comprises the engineered immune cells (e.g., CD70-targeting CAR-T cells) and azacitidine, wherein azacitidine has a concentration of 100 μM or less (e.g., 100 μM or less, 90 μM or less, 80 μM or less, 70 μM or less, 60 μM or less, 50 μM or less, 40 μM or less, 30 μM or less, 20 μM or less, 10 μM or less, 5 μM or less, 1 μM or less) in the composition. In some embodiments, the composition comprises the engineered immune cells (e.g., CD70-targeting CAR-T cells) and azacitidine, wherein azacitidine has a concentration of 100 μM, 90 μM, 80 μM, 70 μM, 60 μM, 50 μM, 40 μM, 30 μM, 20 μM, 10 μM, 5 μM, or 1 μM in the composition.

In some embodiments, the composition is a pharmaceutical composition. In some embodiments, the composition further comprises a pharmaceutically acceptable carrier, excipients or stabilizers typically employed in the art (all of which are termed “excipients”), for example buffering agents, stabilizing agents, preservatives, isotonifiers, non-ionic detergents, antioxidants and/or other miscellaneous additives.

In some embodiments, any one of the engineered immune cells (e.g., CD70-targeting CAR-T cells) described herein or any one of the compositions comprising the engineered immune cells described herein is administered to a subject. Accordingly, some aspects of the present disclosure provide methods of administering to a subject any one of the engineered immune cells (e.g., CD70-targeting CAR-T cells) or the compositions comprising the engineered immune cells (e.g., CD70-targeting CAR-T cells) described herein. In some embodiments, the method is for treating a cancer expressing CD70, and the method comprises administering to a subject in need thereof an effective amount of the engineered immune cells (e.g., CD70-targeting CAR-T cells) or the compositions comprising the engineered immune cells (e.g., CD70-targeting CAR-T cells) described herein.

In some embodiments, the method is for treating a cancer expressing CD70, and the method comprises administering to a subject in need thereof an effective amount of the engineered immune cells (e.g., CD70-targeting CAR-T cells) or the compositions comprising the engineered immune cells (e.g., CD70-targeting CAR-T cells) described herein, and an effective amount of an agent that enhances expression of CD70 in the cancer (e.g., azacitidine or decitabine).

In some embodiments, the engineered immune cells (e.g., CD70-targeting CAR-T cells) and the agent (e.g., azacitidine or decitabine) are administered simultaneously (e.g., the engineered immune cell and the agent are formulated in a composition for administration). In some embodiments, the composition comprises the engineered immune cells (e.g., CD70-targeting CAR-T cells) and azacitidine, wherein azacitidine has a concentration of 100 μM or less (e.g., 100 μM or less, 90 μM or less, 80 μM or less, 70 μM or less, 60 μM or less, 50 μM or less, 40 μM or less, 30 μM or less, 20 μM or less, 10 μM or less, 5 μM or less, 1 μM or less) in the composition. In some embodiments, the composition comprises the engineered immune cells (e.g., CD70-targeting CAR-T cells) and azacitidine, wherein azacitidine has a concentration of 100 μM, 90 μM, 80 μM, 70 μM, 60 μM s, 50 μM, 40 μM, 30 μM, 20 μM, 10 μM, 5 μM, or 1 μM in the composition.

In some embodiments, the engineered immune cells (e.g., CD70-targeting CAR-T cells) and the agent are administered sequentially. In some embodiments, the agent (e.g., azacitidine or decitabine) is administered before the engineered immune cells (e.g., CD70-targeting CAR-T cells) are administered. In some embodiments, there is a waiting period between administering the agent (e.g., azacitidine or decitabine) and administering the engineered immune cell. The waiting period is for the agent (e.g., azacitidine or decitabine) to enhance CD70 expression in the cancer and to clear out of the subject before the engineered immune cells (e.g., CD70-targeting CAR-T cells) are administered. In some embodiments, the waiting period is 3 hours or more (e.g., 3, 4, 5, 6, 7, 8, 9, 10, 12, 24 hours or more).

In some embodiments, the agent (e.g., azacitidine or decitabine) enhances CD70 expression in the cancer by at least 10% (e.g., at least 10%, at least 20%, at least 30%, at least 40%, at least 50%, at least 60%, at least 70%, at least 80%, at least 90%, at least 100%, at least 2-fold, at least 5-fold, at least 10-fold, or more), compared to the same cancer without exposure to the agent (e.g., azacitidine or decitabine).

In some embodiments, administering both the engineered immune cells (e.g., CD70-targeting CAR-T cells) and the agent (e.g., azacitidine or decitabine) to the subject enhances the therapeutic efficacy by at least at least 10% (e.g., at least 10%, at least 20%, at least 30%, at least 40%, at least 50%, at least 60%, at least 70%, at least 80%, at least 90%, at least 100%, at least 2-fold, at least 5-fold, at least 10-fold, or more), compared to when the engineered immune cells (e.g., CD70-targeting CAR-T cells) or the agent (e.g., azacitidine or decitabine) is administered alone. Therapeutic efficacy may be measured by methods known in the art, e.g., clearance of cancer cells, prolonged survival of the subject.

Examples of cancers that express CD70 include, without limitation, bladder cancer, breast invasive carcinoma, cervical cancer, cholangiocarcinoma, colorectal cancer, diffuse large B-cell lymphoma (DLBC), Esophagus, glioblastoma (GBM), head and neck cancer, low-grade gliomas (LGG), liver cancer, lung adeno cancer, melanoma, mesothelioma, ovarian cancer, pancreatic cancer, prostate cancer, sarcoma, stomach cancer, testicular germ cell cancer, thymoma, thyroid cancer, uterine cancer, uveal melanoma, clear cell renal cell carcinoma (ccRCC), chromophobe renal cell carcinoma, papillary renal cell carcinoma (pRCC), acute myeloid leukemia, and adenoid cystic carcinoma (ACC). In some embodiments, the cancer is a myeloid cancer. In some embodiments, the cancer is acute myeloid leukemia.

To practice the methods described herein, an effective amount of the engineered immune cells (e.g., CD70-targeting CAR-T cells) and or the agent that enhances CD70 expression in the cancer (e.g., azacitidine or decitabine) may be administered to a subject via a suitable route (e.g., intravenous infusion). The immune cell population may be mixed with a pharmaceutically acceptable carrier to form a pharmaceutical composition prior to administration, which is also within the scope of the present disclosure.

The subject to be treated may be a mammal (e.g., human, mouse, pig, cow, rat, dog, guinea pig, rabbit, hamster, cat, goat, sheep or monkey). The subject may be suffering from cancer or an immune disorder (e.g., an autoimmune disease).

The term “an effective amount” as used herein refers to the amount of each active agent required to confer therapeutic effect on the subject, either alone or in combination with one or more active agents. Effective amounts vary, as recognized by those skilled in the art, depending on the particular condition being treated, the severity of the condition, individual patient parameters including age, physical condition, size, gender and weight, the duration of treatment, route of administration, excipient usage, co-usage (if any) with other active agents and like factors within the knowledge and expertise of the health practitioner. The quantity to be administered depends on the subject to be treated, including, for example, the capacity of the individual's immune system to produce a cell-mediated immune response. Precise mounts of active ingredient required to be administered depend on the judgment of the practitioner. However, suitable dosage ranges are readily determinable by one skilled in the art.

The term “treating” as used herein refers to the application or administration of a composition including one or more active agents to a subject, who has a target disease, a symptom of the target disease, or a predisposition toward the target disease, with the purpose to cure, heal, alleviate, relieve, alter, remedy, ameliorate, improve, or affect the disease, the symptoms of the disease, or the predisposition toward the disease.

The therapeutic methods described herein may be utilized in conjunction with other types of therapy for cancer, such as chemotherapy, surgery, radiation, gene therapy, and so forth. Such therapies can be administered simultaneously or sequentially (in any order) with the immunotherapy described herein. When co-administered with an additional therapeutic agent, suitable therapeutically effective dosages for each agent may be lowered due to the additive action or synergy.

Non-limiting examples of other anti-cancer therapeutic agents useful for combination with the modified immune cells described herein include, but are not limited to, immune checkpoint inhibitors (e.g., PDL1, PD1, and CTLA4 inhibitors), anti-angiogenic agents (e.g., TNP-470, platelet factor 4, thrombospondin-1, tissue inhibitors of metalloproteases, prolactin, angiostatin, endostatin, bFGF soluble receptor, transforming growth factor beta, interferon alpha, soluble KDR and FLT-1 receptors, and placental proliferin-related protein); a VEGF antagonist (e.g., anti-VEGF antibodies, VEGF variants, soluble VEGF receptor fragments); chemotherapeutic compounds. Exemplary chemotherapeutic compounds include pyrimidine analogs (e.g., 5-fluorouracil, floxuridine, capecitabine, gemcitabine and cytarabine); purine analogs (e.g., fludarabine); folate antagonists (e.g., mercaptopurine and thioguanine); antiproliferative or antimitotic agents, for example, vinca alkaloids; microtubule disruptors such as taxane (e.g., paclitaxel, docetaxel), vincristin, vinblastin, nocodazole, epothilones and navelbine, and epidipodophyllotoxins; DNA damaging agents (e.g., actinomycin, amsacrine, anthracyclines, bleomycin, busulfan, camptothecin, carboplatin, chlorambucil, cisplatin, cyclophosphamide, cytoxan, dactinomycin, daunorubicin, doxorubicin, epirubicin, hexamethyhnelamineoxaliplatin, iphosphamide, melphalan, merchlorehtamine, mitomycin, mitoxantrone, nitrosourea, plicamycin, procarbazine, taxol, taxotere, teniposide, triethylenethiophosphoramide and etoposide).

In some embodiments, radiation, or radiation and chemotherapy are used in combination with the cell populations comprising modified immune cells described herein. Additional useful agents and therapies can be found in Physician's Desk Reference, 59.sup.th edition, (2005), Thomson P D R, Montvale N.J.; Gennaro et al., Eds. Remington's The Science and Practice of Pharmacy 20.sup.th edition, (2000), Lippincott Williams and Wilkins, Baltimore Md.; Braunwald et al., Eds. Harrison's Principles of Internal Medicine, 15.sup.th edition, (2001), McGraw Hill, N.Y.; Berkow et al., Eds. The Merck Manual of Diagnosis and Therapy, (1992), Merck Research Laboratories, Rahway N.J.

### EXAMPLES

While chimeric antigen receptor (CAR) T cell therapy has resulted in dramatic responses in lymphoid malignancies, targeting myeloid diseases remains a substantial challenge in part due to the lack of tumor-specific antigens, and potential for on-target off-tumor toxicities with lineage antigens. Furthermore, efficacy of existing CAR therapies can be compromised via target antigen loss or downregulation. Reported is the first pre-clinical characterization of CAR-T cells targeting the tumor necrosis alpha family member, CD70, for the treatment of acute myeloid leukemia. In addition to consistent expression on myeloid blasts and leukemic stem cells, CD70 has highly restricted expression in healthy human tissues. CD70 CAR T cells demonstrated antigen-specific activation, cytokine production, and cytotoxic activity in models of leukemia in vitro and in vivo. Furthermore, CD70 CARs demonstrated synergy in vivo with the anti-leukemic hypomethylating drug azacitidine, which data showed augments CAR potency via increasing CD70 expression. Results indicate that azacitidine synergizes with CD70 targeted CAR-T cells to treat acute myeloid leukemia.

Acute myeloid leukemia (AML) is the most common acute leukemia in adults. While AML was uniformly fatal half a century ago, it is now curable with intensive chemotherapy in 40% percent of adults¹. While this represents a substantial improvement, there remains a significant unmet clinical need for older and relapsed or refractory patients where cure rates rapidly fall below 10%¹. AML treatment changed little over the fifty years since the advent of intensive “induction” cytotoxic chemotherapy; however, since 2017 there have been eight drugs approved by the FDA for AML, including inhibitors of hedgehog, BCL-2, FLT3, IDH1/2, a CD33 antibody drug conjugate, and a more potent liposomal formulation of induction chemotherapy². While these interventions represent substantial progress, the majority of AML patients still fail to respond or relapse and die from their disease.

The checkpoint blockade revolution has seen dramatic responses in a number of malignancies³ but has had limited success in AML. This discrepancy, is possibly due to low tumor mutational burden in AML, resulting in a dearth of neoantigens for T cells to target, coupled with an immunosuppressive microenvironment characterized by an abundance of myeloid derived suppressor cells (MDSCs),regulatory T cells (T_{regs}), and exhausted effector (T_{eff}) cells⁴⁻⁸. A recent study of T-cell subsets and expression of immune checkpoints in patients with newly diagnosed and relapsed AML identified an enrichment of T_{regs} and exhausted T_{eff} cells in AML patients compared to healthy controls.⁹

Development of adoptive cellular therapy to treat AML has been difficult. The majority of the available surface antigens present on AML blasts are also expressed on many myeloid and stem cell populations, the prolonged ablation of which is not compatible with survival. CARs targeting multiple antigens in AML have been described recently (CD123^{12,13}, cD33^{14,15}, FLT3¹⁶), some of which are currently in phase I clinical trials, though none have been as ideal as CD19 for lymphoid malignancies¹⁷. At least one of these CAR T products targeting CD123 has led to severe side effects including a death in the first patient treated, possibly due to on-target toxicity resulting from CAR targeting of normal vasculature¹⁸.

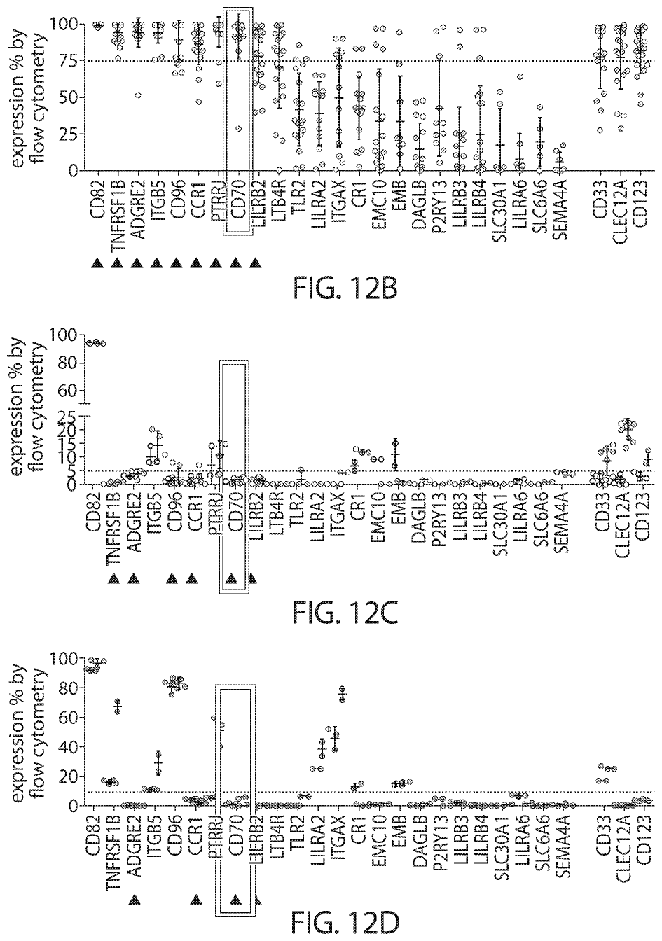

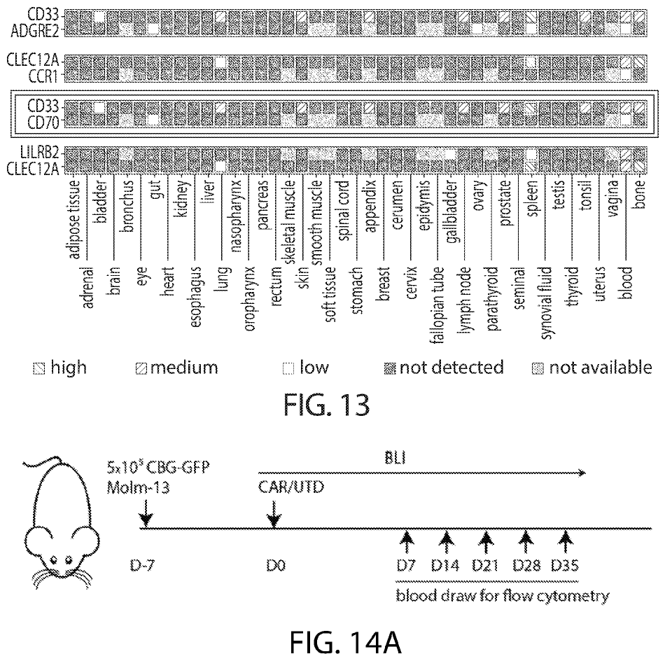

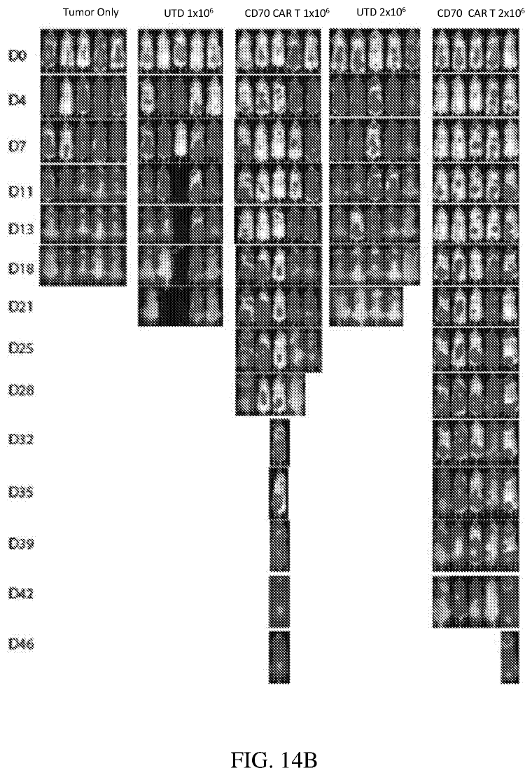

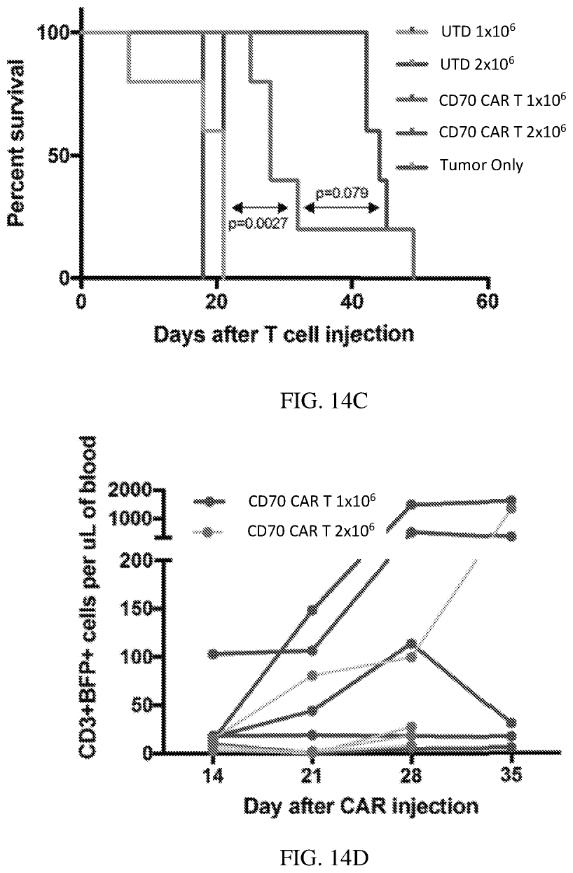

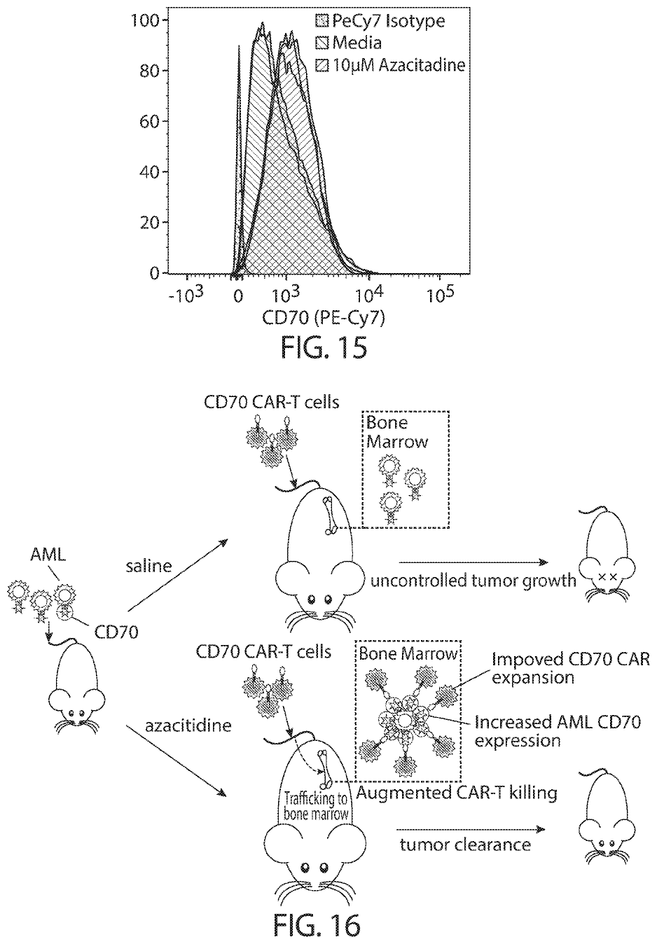

Another antigen expressed by AML that presents a possible target for CAR T cells is CD70 (FIG. 12). CD70 is a tumor necrosis alpha family member that serves as the ligand for CD27, which is involved in T-cell signaling. Expression of CD70 is highly restricted in normal tissues (FIG. 13). This suggests that CAR T cells targeting CD70 may be an attractive option, given that CAR-T cells may have enhanced clinical efficacy over traditional antibody based therapies²².

Given recent findings that even modest decreases in well-chosen target antigen expression may be sufficient to evade CAR killing²⁴, strategies to mitigate potential antigen escape are warranted. Azacitadine (AZA) is currently FDA approved for the treatment of myelodysplastic syndromes, but it (along with decitabine) is also used extensively for the management of patients with AML who are unfit for intensive therapy and is the de facto standard of care²⁷. AZA and its deoxy derivative, decitabine, are nucleoside analogues which inhibit DNA methyltransferase resulting in the hypomethylation of DNA and cause direct cytotoxicity by integrating into nucleic acids²⁸. AZA and decitabine are part of a larger category of drugs referred to as demethylation agents. Recently, AZA was shown to cause hypomethylation of the CD70 promoter, resulting in increased CD70 surface expression in solid tumor cell lines as well as primary AML blasts^{21,29}.

In this work, CD70 targeted CAR T cells were developed and tested them alone and in combination with AZA using in vitro and in vivo models of AML. Results demonstrated significant CAR activity against AML in vitro and in vivo. Furthermore, results showed combining azacytidine and CD70 CAR is a feasible combinatorial approach to enhance efficacy and increase CD70 target antigen density. With this combined approach, a modest increase in tumor antigen expression caused by azacytidine was sufficient to enhance CAR killing in vivo and provided durable clearance of tumor in an exceptionally aggressive tumor model. Data also showed that CD70 CAR-T cells maintained effector functions in vitro after being exposed to clinically relevant azacitidine concentrations. In summary, azacitidine synergized with a novel CAR-T cell therapy and treated AML, a cancer that has traditionally been exceptionally difficult to target.

### Methods

### Construction of CARs and T-Cell Culture Transduction

CD70 CAR construct was synthesized and cloned into a third-generation lentiviral plasmid backbone using human EF-1α promoter. The extracellular and transmembrane portions of CD27 were ligated to the 4-1BB costimulatory and CD3t signaling domains to generate a ligand-based CAR. Blue Fluorescent protein (BFP) was appended to the CAR via a self-cleaving peptide sequence to assess transduction. Human T cells were purified from healthy donor leukopaks (via kit from Stem Cell Technologies, Catalog #15061) purchased from the Massachusetts General Hospital blood bank via an institutional review board-approved protocol.

### Cellular Cytotoxicity and Cytokine Assays

Cytotoxicity was assessed via co-culture of CAR-T cells with click beetle green (CBG) luciferase-expressing tumor targets at the indicated ratios for approximately 16 hours. Luciferase activity was measured using a Synergy Neo2 microplate reader from Biotek. Soluble cytokines were assessed at approximately 16 hours after 1:1 co-culture of CAR-T cells with tumor targets.

### In Vivo Experiments

All animal research was conducted in accordance with Federal and Institutional Animal Care and Use Committee requirements within a protocol approved at Massachusetts General Hospital.

### Cell Lines and Molecular Reagents

Molm13 was obtained from the American Type Culture Collection and maintained under conditions as outlined by the supplier. Where indicated, Molm13 lines were transduced to express click beetle green (CBG) luciferase and enhanced GFP (eGFP) and sorted on a BD FACSAria to obtain a clonal population of transduced cells. CD70 null cells were generated via use of the following CD70 CRISPR guide from the Brunello library “GAGCTGCAGCTGAATCACAC”³⁰. DNA guides were purchased from integrated DNA technologies (IDT) and converted to RNA via the HiScribe T7 Quick High Yield RNA Synthesis Kit (New England Biolabs, E2050S). After guide RNA and Cas9 protein electroporation into Molm13 cells, single cell clones were established via sorting on a BD FACSAria and monitored for similar proliferative capacity to parental lines. Finally, one clone was chosen to engineer increasing levels of truncated CD70 protein by lentiviral transduction. These Molm13 null, trCD70 transduced lines were then sorted via BD FACSAria for only CD70 expressing cells to establish lines of variable expression.

### Real-Time Cytotoxicity Assay

Cell index was recorded as a measure of impedance using the xCELLigence RTCA SP instrument (ACEA biosciences). After confirming robust Molm13 CD71 protein expression, target cells were mobilized on the plate bottom by pre-coating the wells with CD71 antibody (BioLegend, 334102). 125,000 Molm13 cells per well were then plated for 28 hours followed by administration of 125,000 CAR-T cells. Cell index was tracked for 96 hours.

### Plate Bound Antigen Activation Assay

Recombinant Human CD70 (CD70, R&D Systems 9328-CL-100) was plated for 3 hours in a 96 well plate at 1 ug/well. After washing in PBS, trD27 or CAR T cells were added for 12 hours followed by flow cytometric staining for CD69.

### Primary Human T Cell Culture

Human T-cells were activated using CD3/CD28 Dynabeads (LifeTechnologies) on Day 0, followed by transduction with a lentiviral vector encoding the CAR on day 1 (24 hours later). T cells were cultured in RPMI media containing 10% fetal bovine serum with 20 IU/mL of recombinant human IL-2, penicillin, and streptomycin. T cells were debeaded on day 7 and cryopreserved on day 14.

### Flow Cytometry and IHC

The following antibody clones targeting their respective antigens were used for flow cytometric analysis where indicated: CD70 (113-16, Biolegend), PeCy7 isotype (MOPC-21, Biolegend), CD69 (FN50, Biolegend), CD107a (H4A3, Biolegend), mouse TER-119 (TER-119, Biolegend), mouse NK-1.1 (PK136, Biolegend), mouse Ly-6G/Ly-6C (Gr-1, Biolegend), mouse CD11b (Biolegend). In general, cells were stained for fifteen minutes in the dark at 4 degrees Celsius and washed in PBS with 2% FBS. When used, DAPI was added to establish live versus dead separation. Trucount tubes (BD Biosciences, 340334) were used for murine blood CAR quantification according to kit instructions. Quantum Simply Cellular beads (Bangs Laboratories, 815) were used to quantify CD70 surface expression.

For IHC, murine femurs were washed in PBS and then incubated overnight in 4% paraformaldehyde (PF Thermo-Fisher Scientific AAJ19943K2), followed by an overnight incubation in Cal-ex decalcifier (Fisher Scientific, CS510-1D) and then storage in 70% ethanol until staining. Antibody clones for IHC included the following: CD3 (2GV6, Roche) and CD45 (D9M81, Cell Signaling Technology).

### In Vivo Experiments

NOD-SCID-γ chain−/− (NSG) (Jackson Laboratories) mice were engrafted with Molm13 cell lines as described for the individual experiments. Mice were maintained at the MGH Center for Cancer Research and all care and conducted experiments were carried out using protocols approved by the Massachusetts General Hospital Institutional Animal Care and Use Committee. Due to instability in solution, Azacitidine (Sigma-Aldrich catalog #A2385-100MG) stock solutions were made fresh daily and administered via intraperitoneal injection. Cryopreserved CD70 CAR T cells or untransduced T cells were injected intravenously via tail vein at the indicated time points. Tumor burden was monitored via bioluminescence following intraperitoneal injection of D-luciferin substrate solution. AMI spectral imaging was used to perform the image capture and IDL software v. 4.3.1 was used for analysis. Animals were euthanized per the experimental protocol or when they met a priori defined endpoints by IACUC.

### Statistical Analysis

Data were presented as mean±standard deviation or as standard error of the mean with statistically significant differences determined by tests as indicated in FIG. legends. Significance was considered at a P<0.05. Data was analyzed using GraphPad Prism 7 (Version 8.3). Specific lysis was calculated via the following equation % specific lysis=(total luciferase/target cells only luciferase)×100%. Luminex (Luminex Corp, FLEXMAP 3D) was used to analyze cell-free supernatants for cytokine production according to the manufacturer's recommendation in technical duplicates. All assays were performed in biologic duplicates or triplicates based on the number of unique donor T cells tested.

### Results

### Generation of CD70 CAR-T Cells

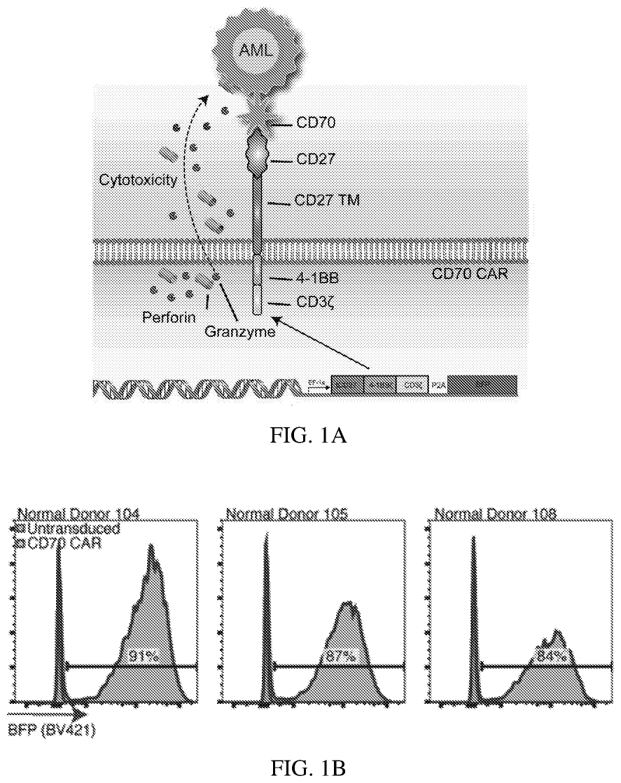

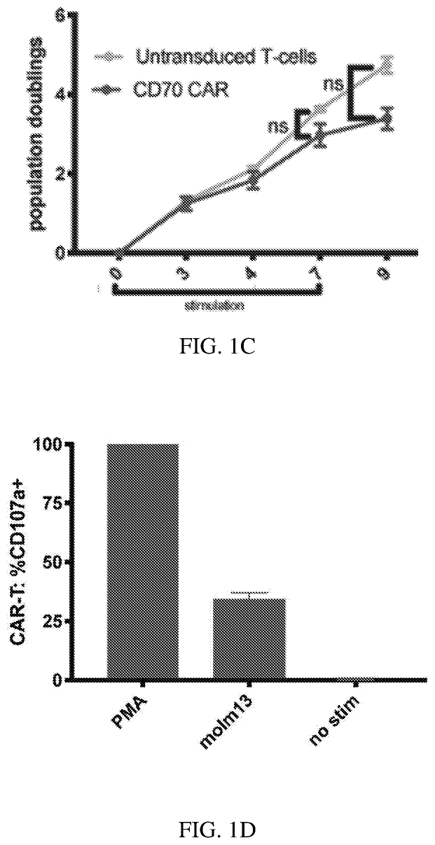

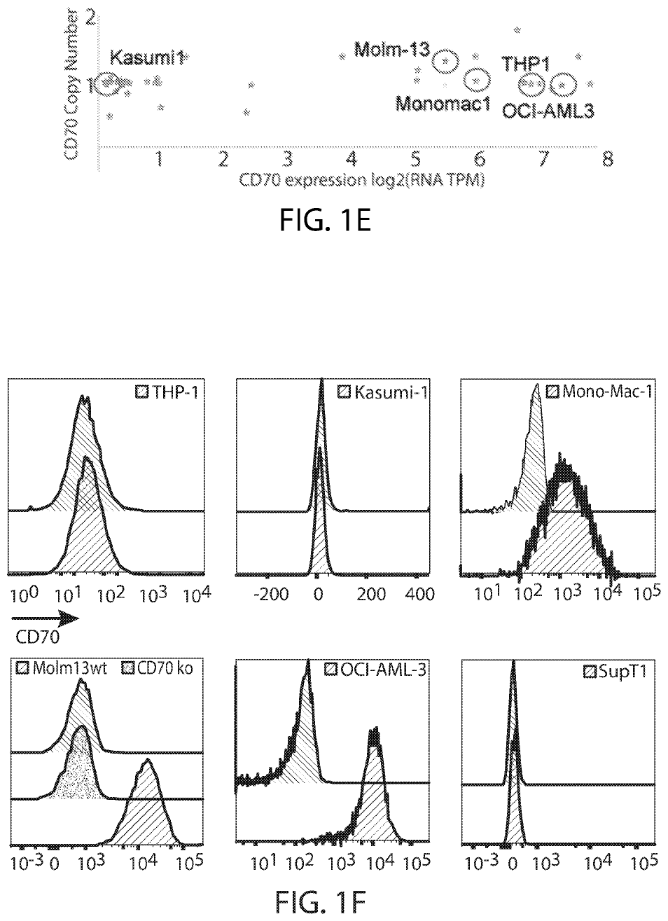

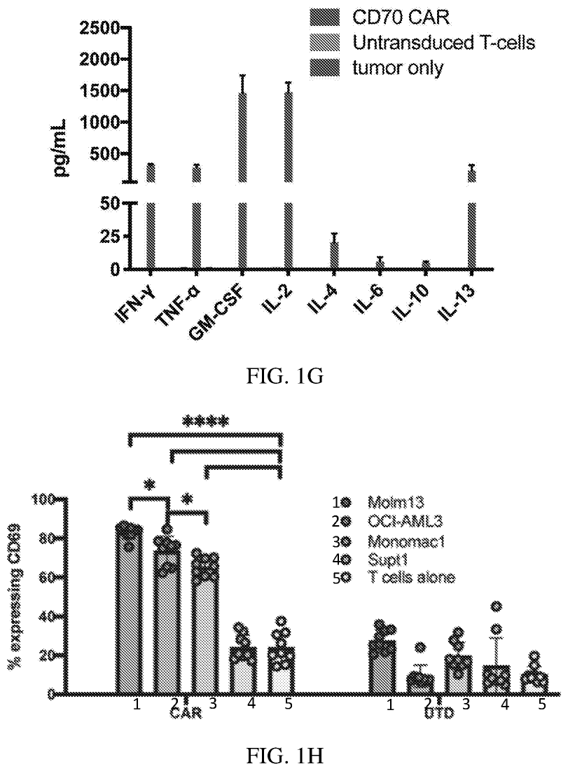

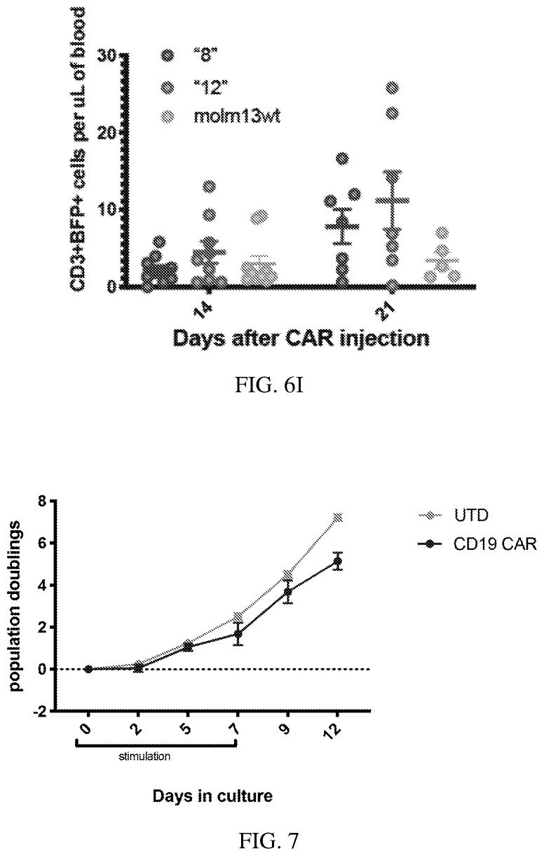

CD70 targeted CAR constructs were generated by fusing the extracellular and transmembrane portions of CD27 to the 41BB and CD3ζ intracellular signaling domains. The blue fluorescent protein (BFP) reporter gene was included after a 2A ribosomal skip sequence to assess for lentiviral transduction (FIG. 1A). With the CD70 CAR, high transduction efficiencies (between 84-91%) were achieved in healthy donor T cells (FIG. 1B), possibly due to the smaller size of a ligand-based construct as compared to traditional antibody-based CAR designs. Although activated T cells express CD70, and there was potential for fratricide during T cell manufacturing, CD70 CAR-T cell expansion was not inferior to untransduced T cells (UTD) by day 9 (FIG. 1C) and was comparable to the CD19 41BB CAR construct control (FIG. 7).

### CD27-Based CAR T Cells Exhibited Robust Effector Functions in Response to CD70 Positive Target Cells.

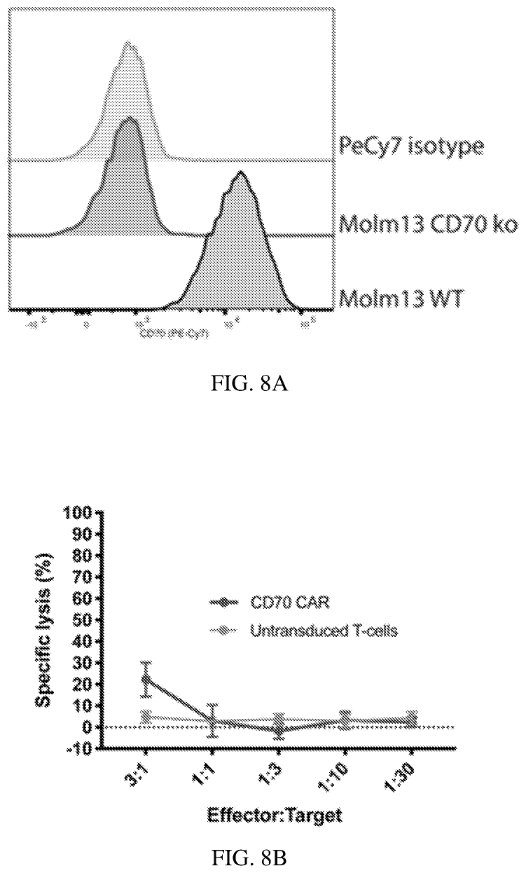

CD70-targeted CARs ability to degranulate was assayed in response to an AML cell line, Molm13 (FIG. 1D). To extend these findings to other AML cell lines and characterize antigen density requirements, bulk RNA expression and copy number data was analyzed for CD70 obtained via depmap (https://depmap.org/portal/) for all characterized AML cell lines in the database (FIG. 1E). Cell lines chosen and utilized in this work, with a range of expression levels, are highlighted. CD70 expression in AML cell lines at the protein level was confirmed by flow cytometry (using the T cell line, SupT1, as a negative control) (FIG. 1F). When CD70-targeted CAR-T cells were co-cultured 1:1 with AML cells, the CAR-T cells produced Th1-type cytokines relative to UTD T cells (FIG. 1G). CD70 expressing AML cell lines induced expression of the CAR-T activation marker, CD69, on co-culture with CD70 CAR-T cells (FIG. 1H). Finally, to assess the in vitro cytotoxicity of CD70-targeted CAR-T cells against leukemia, an overnight cytotoxicity assay against AML cell lines was performed, and antigen-density-responsive cytotoxicity across the AML cells lines was observed (FIG. 1I). CD70-targeted CAR-T cells exhibited minimal cytolytic activity against CD70 knockout Molm13 targets (FIG. 8A), further confirming antigen specificity. Consistent with expectations, Molm13 treatment with UTD CAR-T cells caused minimal specific lysis suggesting the CD70 CAR-T was critical for targeting AML (FIG. 8B).

### CD70 Targeted CAR T Cells Mediated In Vivo Tumor Control.

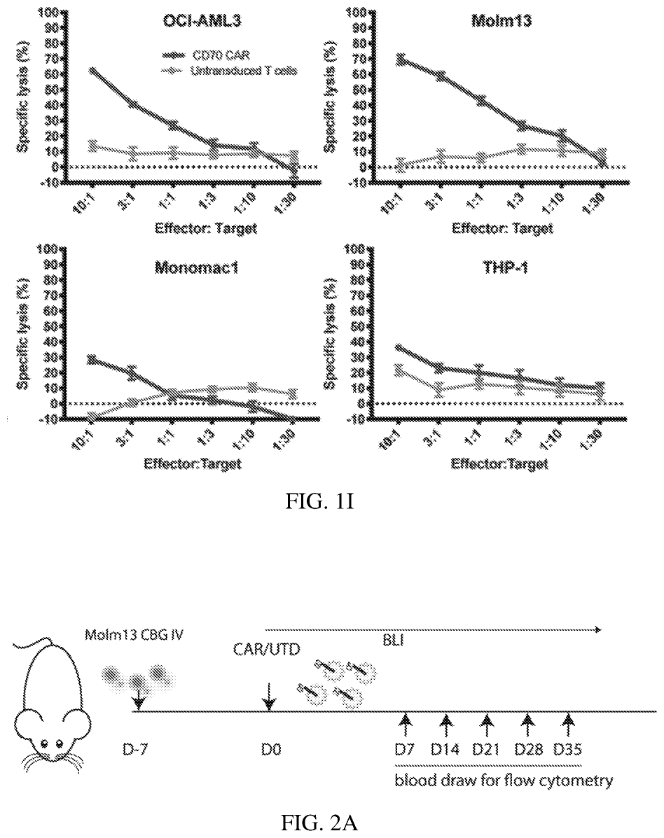

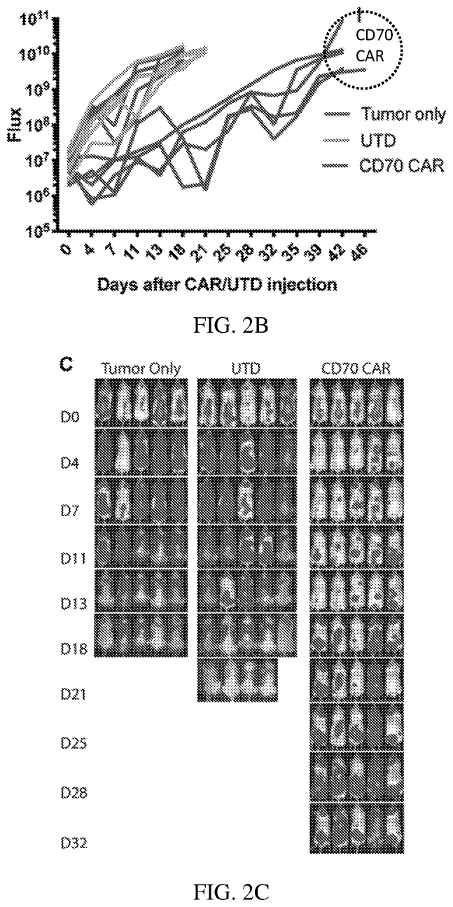

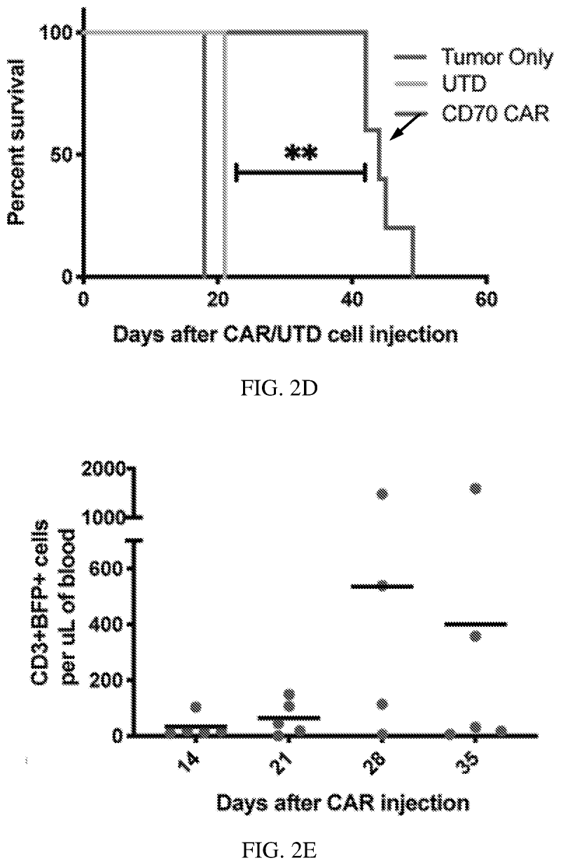

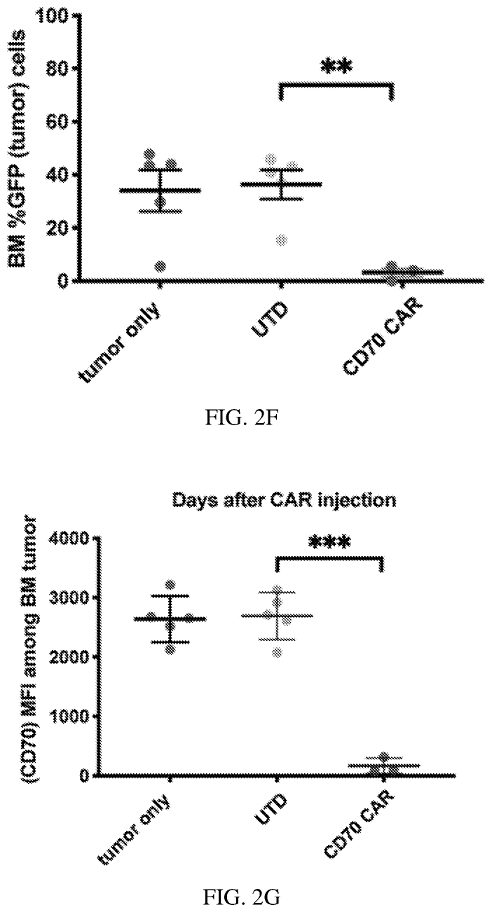

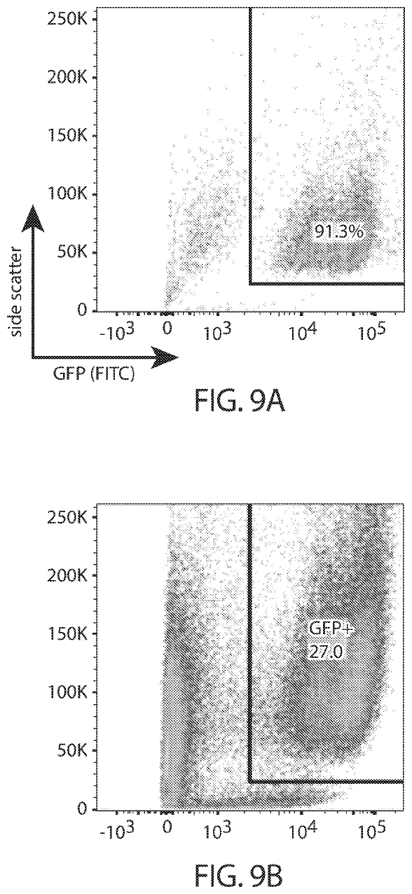

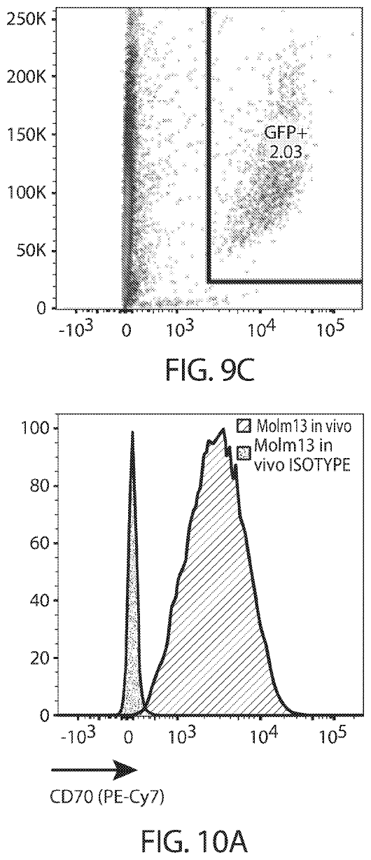

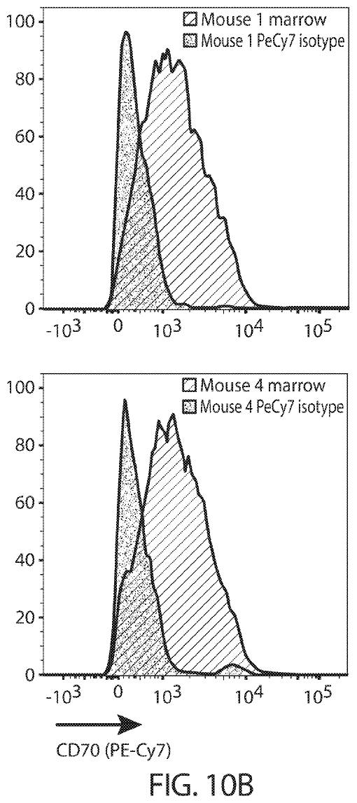

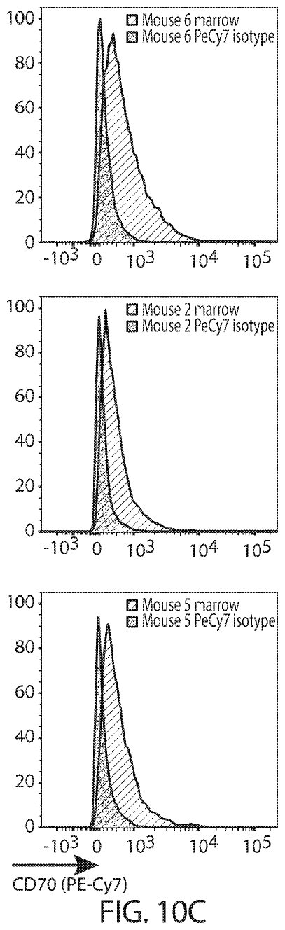

Next experiments were used to determine if CD70-targeted CAR T cells were effective in xenograft models of AML in vivo. NSG mice were injected intravenously with 5×10⁵ AML cells. Seven days after injection, disease burden was assessed by bioluminescence imaging (BLI). The animals were randomized based on total body flux to control for starting tumor burden, and injected with 2×10⁶ CD70-targeted CAR cells or the equivalent number of UTD T cells (FIG. 2A). CAR-T treated mice demonstrated improved tumor control as measured by BLI (FIG. 2B, FIG. 2C) and improved survival (FIG. 2D) compared to those that received UTD T cells. CAR-T expansion was observed at days 28 and 35 (FIG. 2E). Tumor burden in the bone marrow was significantly lower in the CAR-T treated mice compared to untreated or UTD T cell treated mice. (FIG. 2F and FIG. 9). Given the minimal bone marrow involvement observed, mortality appeared to be driven by extramedullary disease, which is of unclear significance in AML NSG xenograft models³⁰. Interestingly, residual tumor cells had significantly less CD70 expression in the CD70-targeted CAR treated mice (FIG. 2G).

### Addition of Azacitidine to CD70 CAR-T Cell Therapy Facilitates Eradicate In Vivo AML.

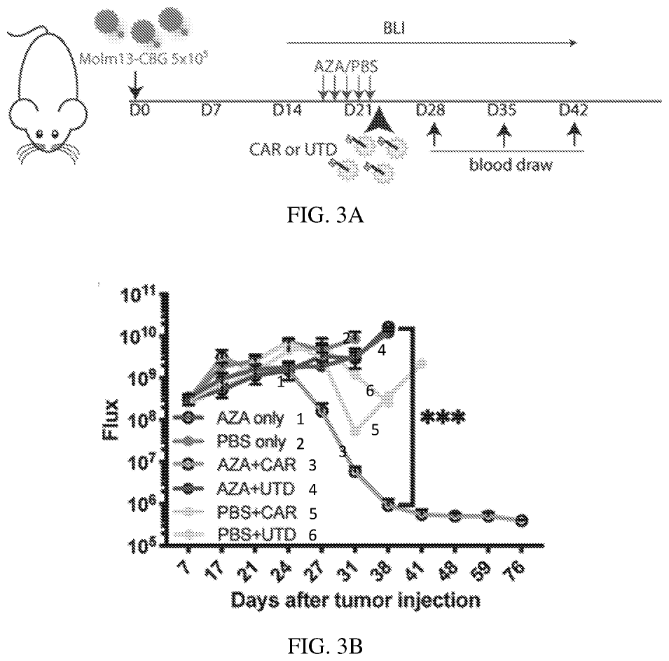

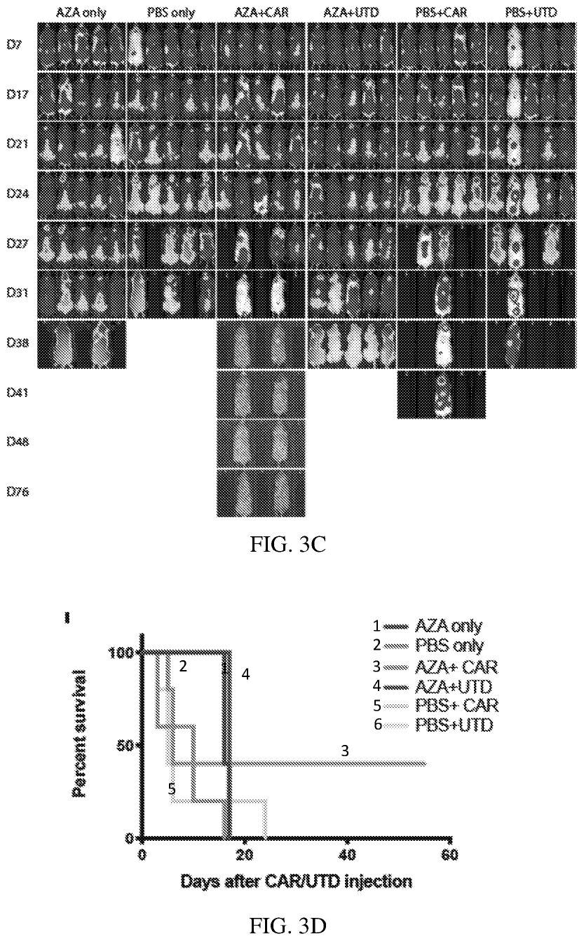

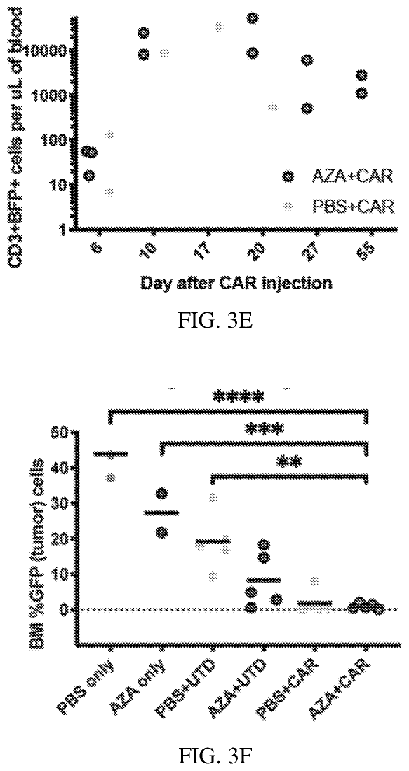

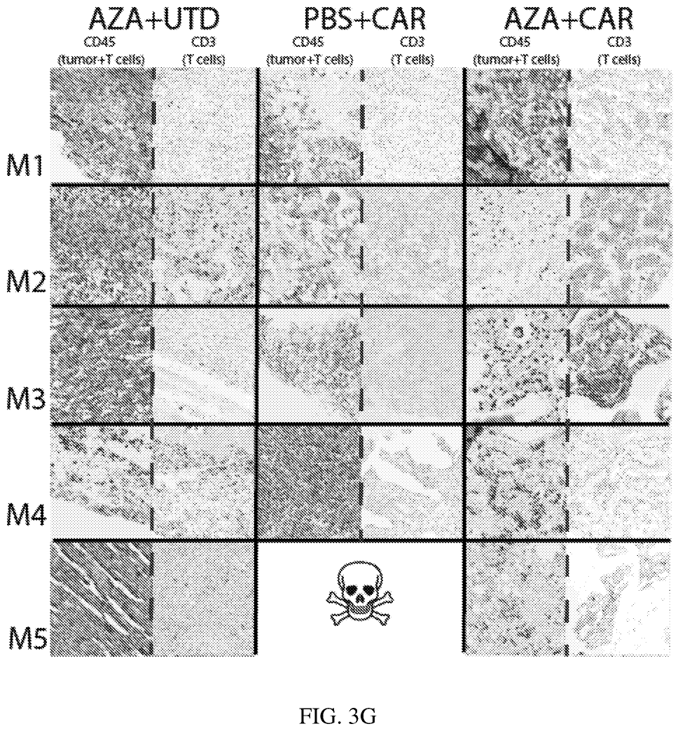

Since CD70 CAR-T cells improved tumor response but did not lead to durable tumor control in the in vivo model, an alternative means was sought to improve CD70 CAR-T cell potency. It was hypothesized that combining AZA with CD70 CAR-T cells might be synergistic for the treatment of AML. To test this hypothesis, NSG mice were injected with tumor and allowed an extended engraftment period to ensure tumor burden was in excess of the amount that could be controlled via the limited, single-agent tumoricidal effects of AZA. Starting on day +18, mice received intraperitoneal injections of AZA or vehicle (phosphate buffered saline, PBS) for five days (FIG. 3A). Following the final AZA injection on day +22, a washout period of 6 hours (representing ˜8 times the half-life for the normal human subcutaneous dose³¹) was allowed to ensure that residual AZA would not modulate the subsequently administered T-cells and confound interpretation of its effects on Molm13. Mice then received CD70 CAR-T cells, UTD T cells, or no injection. The extended tumor engraftment resulted in an aggressive tumor model comprising of 22 days of high tumor burden engraftment (with only scant partial treatment via AZA for 5 days in some of the animals). The PBS and PBS+UTD treated mice succumbed to disease by day 20. Compared to the PBS and PBS+UTD treated mice, the AZA and AZA+UTD treated groups appeared to have evidence of a slight treatment effect. Finally, while, several PBS+CAR treated mice showed improved tumor control compared to the untreated mice, only the AZA+CAR treated mice had prolonged survival without any detectable tumor. These mice were only sacrificed at day 76 after meeting IACUC standards for xenogeneic graft versus host disease (FIG. 3B, FIG. 3C, FIG. 3D). For context, most Molm13 NSG tumor models treated with adoptive cellular therapy typically inject substantially less cells for engraftment, treat much earlier (day 3-14), and with many more CARs (5-10 million) and have generally been non curative³²⁻³⁴. Results showed that CAR expansion was robust and persistent, as peak levels were several logs greater than those in FIG. 2, demonstrating the ability of CD70 CAR-T cells to proliferate in a tumor-burden dependent fashion (FIG. 3E). Flow cytometric evaluation of bone marrow from AZA+CAR treated mice revealed nearly absent tumor cells in all group members (FIG. 3F). A complementary technique was used to verify tumor burden and CAR-T cell expansion. Immunohistochemical evaluation of the bones revealed abundant human CD45 staining in the AZA+UTD and AZA+PBS groups (FIG. 3G). Conversely, human CD45 staining was scattered in the AZA+CAR group in a background of normal appearing, organized hematopoiesis. To determine if these were cancer cells or infiltrating CAR-T cells, human CD3 was stained. In the context of marked human CD3 expression in the same mice, these scattered cells reflect infiltrating CAR-T cells which were not seen in the PBS+CAR group.

### Azacitidine Exposure Resulted in Increased CD70 Expression by Molm13 Both In Vitro and In Vivo

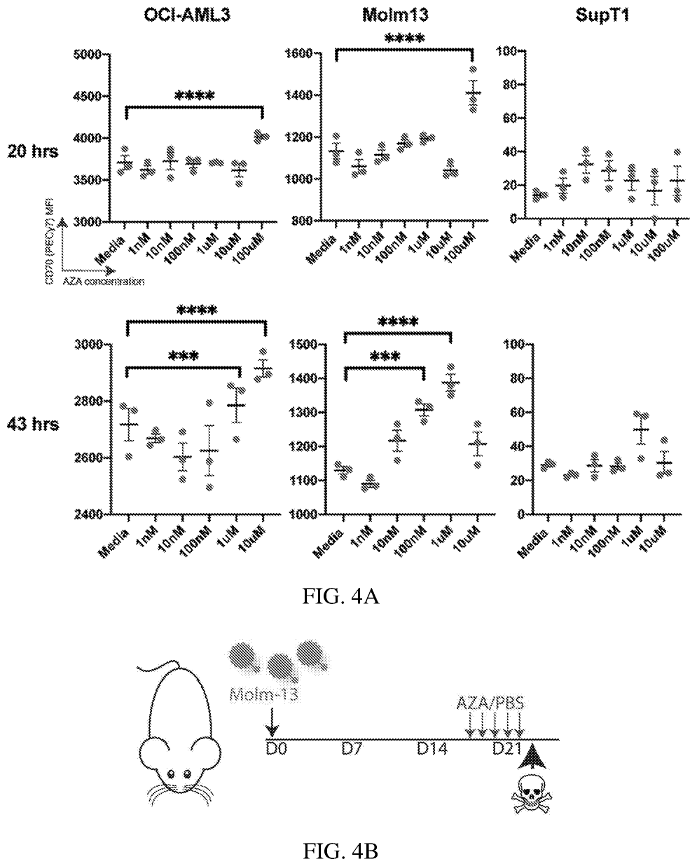

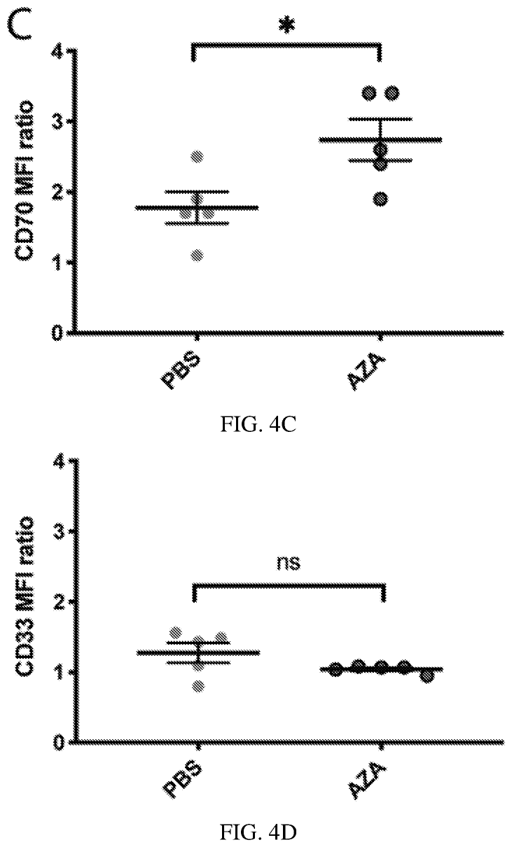

The direct effect of AZA on tumor CD70 expression was then studied. To determine if, and to what extent AZA increases CD70 expression, OCI-AML3 and Molm13 AML lines and the T-cell line, SupT1, were incubated with AZA for 20 and 44 hours, after which CD70 was measured by flow cytometry. Incubation with AZA resulted in an increase in CD70 expression at higher concentrations in the AML lines, but not the T-cell line (FIG. 4A and FIG. 4B). Notably the range of these concentrations are inclusive of the peak levels of AZA detected in humans after subcutaneous injection (3 μM³⁵), suggesting these concentrations are clinically relevant.

Next the effect of AZA on CD70 expression in AML in vivo was confirmed. After a prolonged engraftment of AML in xenografted mice for 18 days, AZA or vehicle (PBS) was administered IP for five days before sacrificing the mice on day 22. Femur aspirates were analyzed for tumor cells and their CD70 expression via flow cytometry (FIG. 4C). CD70 expression level was compared to isotype controls for each mouse. The median fluorescent intensity (MFI) ratio of CD70 relative to the isotype was significantly elevated in the mice that had received five days of AZA compared to PBS control (FIG. 4C, Supplemental FIG. 4). This effect appeared to be specific to CD70 expression, as an increase in the canonical myeloid marker CD33 was not observed with AZA treatment (FIG. 4D).

### CD70 CAR-T Cells Maintained Effector Functions in the Presence of Therapeutic Levels of AZA.

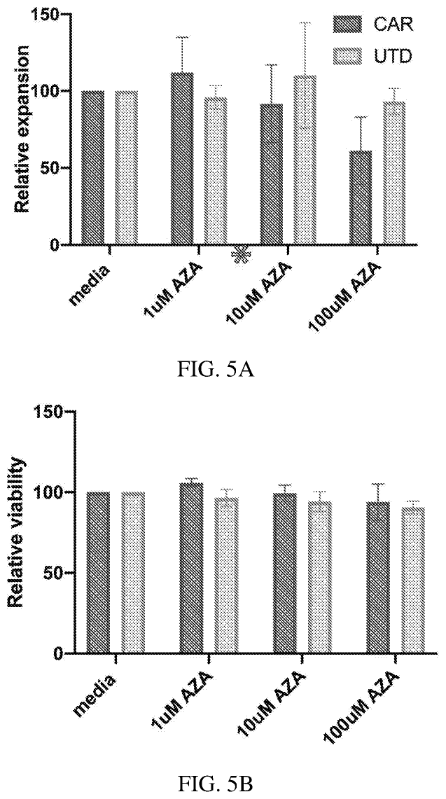

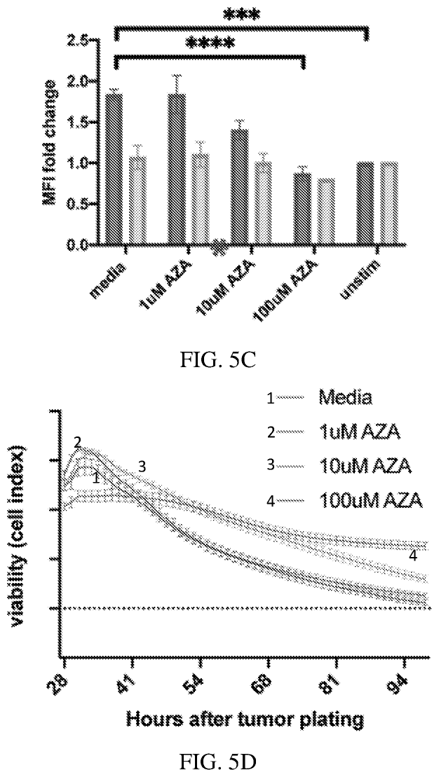

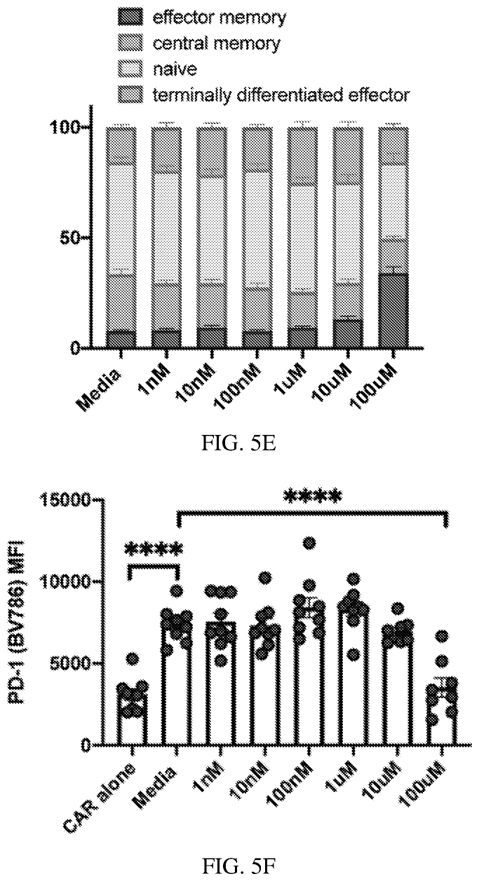

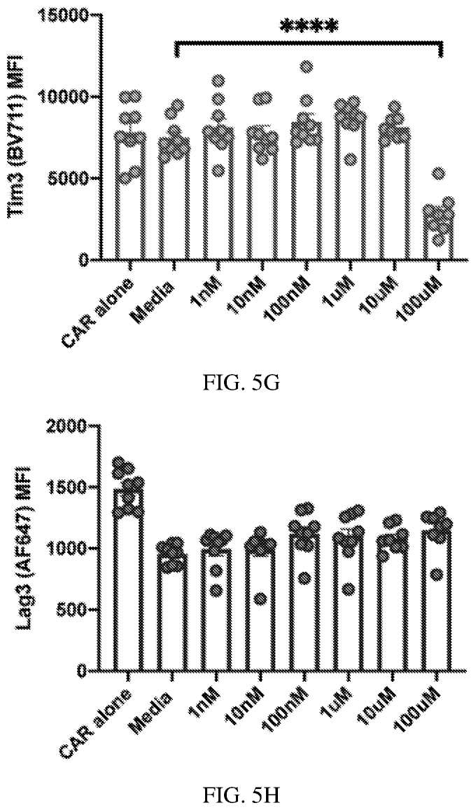

Since AZA exerts its therapeutic effects in AML through inhibition of DNA methyltransferase, it was determined if clinically relevant concentrations of AZA would also affect CD70 CAR-T cells or impair their function. CD70 CAR-T cells generated from three healthy donors were exposed to increasing concentrations of AZA for 24 hours in the presence of IL-2. The same number of CAR-T cells survived, and viability was preserved across all conditions (FIG. 5B). Next, CD70 CAR-T cell activation in the presence of AZA was assessed. CD70 CAR-T cells were incubated in AZA, washed, and then exposed to plate-bound CD70 antigen overnight. Only when CAR-T cells were exposed to a supratherapeutic concentration (100 μM) of AZA was there a significant reduction in activation compared to control (FIG. 5C). Finally, CD70 CAR-T cells were incubated with increasing concentrations of AZA for 24 hours washed, and then incubated with AML targets to measure real time cytotoxicity. CD70-targeted CAR-T cells treated in 1 μM AZA demonstrated persistent cytotoxicity with no statistically significant difference from control after 96 hours (FIG. 5D). Next, the effects of AZA on CD70 CAR T cell memory phenotype and activation/exhaustion markers were quantified. CD70 CAR-T cells were co-cultured with Molm13 AML cells for two days and T-cell subset markers were assessed by flow cytometry. At the highest concentrations of AZA exposure, an increase in effector memory phenotypes (FIG. 5E) and decreases in PD-1 (FIG. 5F) and Tim 3 (FIG. 5G) but not Lag3 (FIG. 5H) were observed, indicating no enhancement of an exhaustion phenotype.

### Increased CD70 Expression Resulted in Improved In Vitro Activation and In Vivo Clearance by CD70 CAR-T Cells.

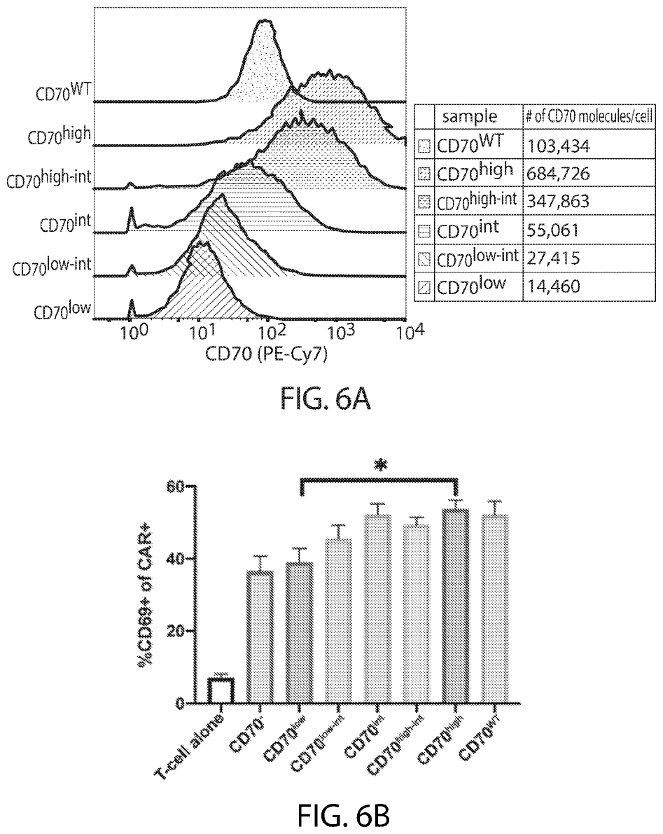

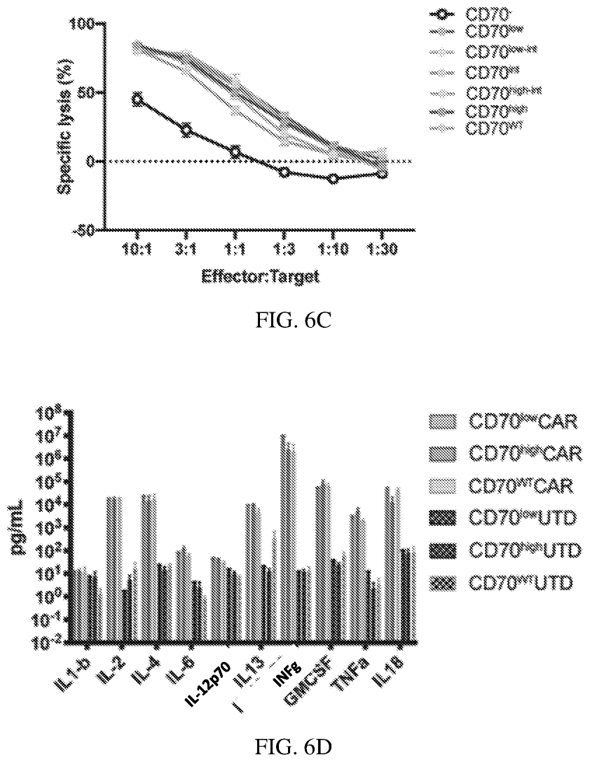

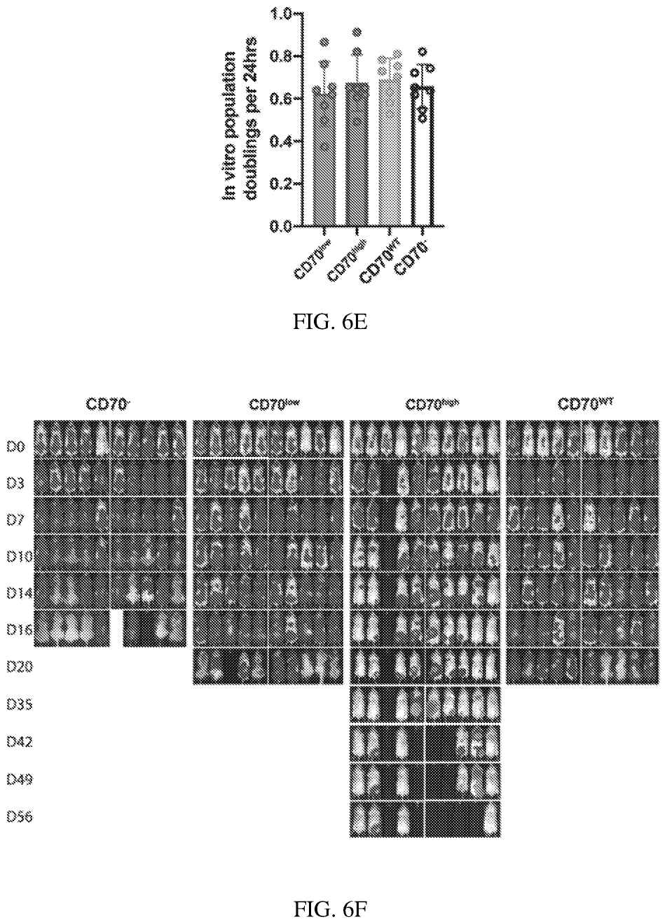

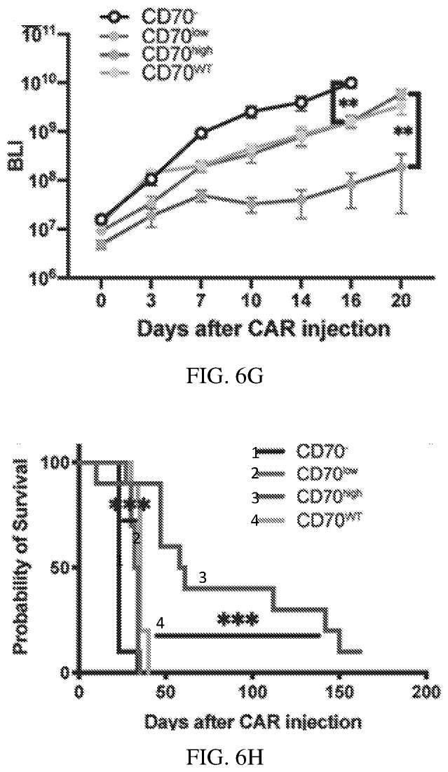

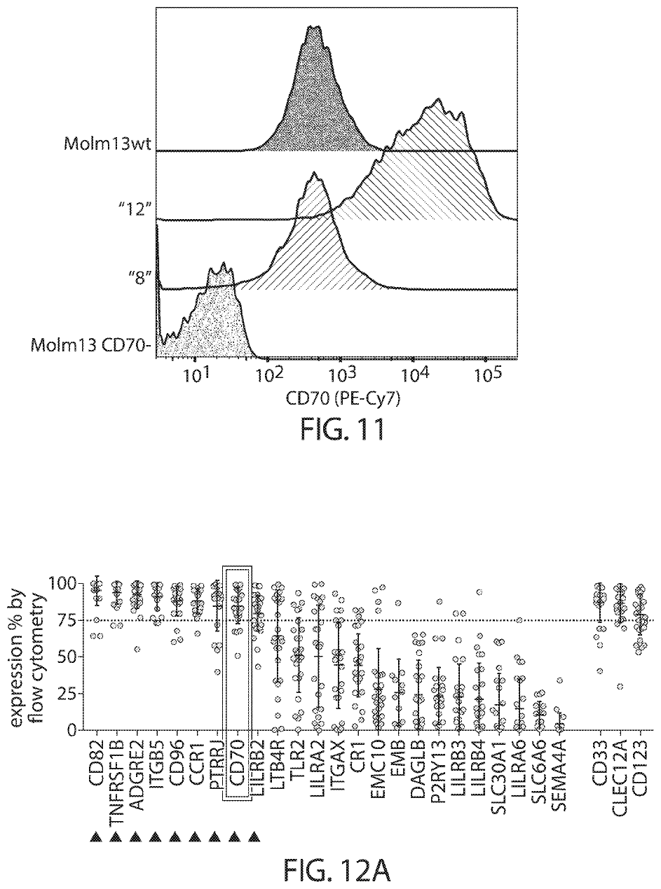

Next, the effect of antigen density on CD70 CAR-T cell function independent of AZA was determined. CD70 null Molm13 cells were generated via CRISPR deletion of CD70 and then transduced with variable levels of lentivirus coding for a truncated, membrane-bound CD70 protein, generating five new cell lines with varying levels of CD70 expression. CD70 protein under the regulator control of the human EF1 alpha promoter. To avoid signaling downstream of CD70, the truncated protein lacked an intracellular signaling domain (FIG. 6A, FIG. 11). When cocultured with CD70 CAR-T cells, the degree of CD70 CAR-T cell activation was significantly different only between the highest and lowest CD70 expressors (FIG. 6B). However, there was no discernable difference in CD70 CAR-T cell killing of these lines (FIG. 6C) or in cytokine generation after 18 hours of incubation at a 1:1 ratio (FIG. 6D).

Traditional in vitro functional assays might be insufficient to distinguish subtle differences in antigen density and, thus, selected several lines for in vivo assessment. Notably, the fold-increase in expression of CD70 on cell line “12” relative to wild type (WT) Molm13 approximates the same fold-change that has been shown to occur in AZA treated primary patient blasts²¹. Prior to in vivo engraftment, data confirmed that there were no differences between in vitro population doubling times among the various tumor lines (FIG. 6E). Mice were injected with cells of each tumor line and treated with CD70 CAR-T cells as in FIG. 3A. This was expected to be a non-curative CAR dose and, therefore, stratify differences between groups. Mice bearing CD70- AML were quickly overtaken with tumor burden and succumbed to disease on day 16, while those bearing CD70high AML had improved tumor control and lived significantly longer (FIG. 6F, FIG. 6G). Mice bearing wild-type tumors lived longer than those with CD70 knockout tumors, while mice harboring CD70 high tumors had substantially prolonged survival with 4/10 surviving over 100 days (FIG. 6H). Mice with CD70high tumors also had superior CD70-targeted CAR expansion than wild-type tumors by day 21 (FIG. 61).

### Discussion

Of note, the CD70 CAR presented in this study has several potential advantages over antibody-based constructs. Many currently available CAR designs are based on murine single chain variable fragment (ScFv) clones such as FMC63 for CD19. These are known to drive immunogenic responses that potentially limit persistence in patients⁴⁶. This construct uses the natural ligand for CD70, and thus, is inherently human and not immunogenic. Secondly, the smaller size of this ligand-based construct relative to ScFv constructs results in a smaller genetic payload, and improved transduction efficiency which potentially translates to improved manufacturing parameters.

Importantly, CD70 is expressed on a small subset of immune cells including antigen presenting cells and activated T-cells which leads to theoretical concerns of fratricide and immune targeting¹⁹. However, difficulties with expansion or efficacy in vitro or in vivo were not observed in this study²³.

Multiple mechanisms of failure to CAR-T cell therapy have been elucidated including intrinsic T-cell deficits⁴⁸⁻⁵⁰, antigen loss⁴⁷, and antigen down-regulation^{25,47}. One promising strategy to mitigate antigen loss and down regulation has been the use of pharmacologic agents that can increase target antigen expression like was done here with AZA. While AZA is known to have pleotropic effects as a hypomethylator, results showed that its ability to increase protein expression is not universal and not applicable to all myeloid targets.

Results showed that a ligand-based CD70-targeted CAR-T cell construct is effective against in vitro and in vivo models of AML and that the anti-leukemic drug, AZA, increases expression levels of CD70 and its administration, in combination with CD70 CARs, is requisite for clearance of an aggressive AML model (FIG. 16). Independently, results demonstrated that higher antigen density significantly augments CD70 CAR function in vivo. Finally, results identified a therapeutic window in which CAR-T cells continue to function after exposure to clinically relevant concentrations of AZA. This strategy leverages the existing anti-tumor effects of AZA, while incorporating and augmenting CAR-T cell therapy.

TABLE 1
Amino Acid Sequences

| | Amino Acid Sequence |
|---|---|
| CD70 CAR | ATPAPKSCPERHYWAQGKLCCQMCEPGTFLVKDCDQHRKAAQ |
| extracellular target | CDPCIPGVSFSPDHHTRPHCESCRHCNSGLLVRNCTITANAECAC |
| binding domain | RNGWQCRDKECTECD (SEQ ID NO: 1) |
| CD70 CAR 1 | ATPAPKSCPERHYWAQGKLCCQMCEPGTFLVKDCDQHRKAAQ |
| | CDPCIPGVSFSPDHHTRPHCESCRHCNSGLLVRNCTITANAECAC |
| | RNGWQCRDKECTECDPLPNPSLTARSSQALSPHPQPTHLPYVSE |
| | MLEARTAGHMQTLADFRQLPARTLSTHWPPQRSLCSSDFIRILVI |
| | FSGMFLVFTLAGALFLKRGRKKLLYIFKQPFMRPVQTTQEEDGC |
| | SCRFPEEEEGGCELRVKFSRSADAPAYQQGQNQLYNELNLGRRE |
| | EYDVLDKRRGRDPEMGGKPRRKNPQEGLYNELQKDKMAEAYS |
| | EIGMKGERRRGKGHDGLYQGLSTATKDTYDALHMQALPPR |
| | (SEQ ID NO: 2) |
| CD70 CAR 2 | EQKLISEEDLATPAPKSCPERHYWAQGKLCCQMCEPGTFLVKDC |
| | DQHRKAAQCDPCIPGVSFSPDHHTRPHCESCRHCNSGLLVRNCTI |
| | TANAECACRNGWQCRDKECTECDPLPNPSLTARSSQALSPHPQP |
| | THLPYVSEMLEARTAGHMQTLADFRQLPARTLSTHWPPQRSLCS |
| | SDFIRILVIFSGMFLVFTLAGALFLKRGRKKLLYIFKQPFMRPVQT |
| | TQEEDGCSCRFPEEEEGGCELRVKFSRSADAPAYQQGQNQLYNE |
| | LNLGRREEYDVLDKRRGRDPEMGGKPRRKNPQEGLYNELQKD |
| | KMAEAYSEIGMKGERRRGKGHDGLYQGLSTATKDTYDALHMQ |
| | ALPPR (SEQ ID NO: 3) |
| CD70 CAR 3 | ATPAPKSCPERHYWAQGKLCCQMCEPGTFLVKDCDQHRKAAQ |
| | CDPCIPGVSFSPDHHTRPHCESCRHCNSGLLVRNCTITANAECAC |
| | RNGWQCRDKECTECDTTTPAPRPPTPAPTIASQPLSLRPEACRPA |
| | AGGAVHTRGLDFACDIYIWAPLAGTCGVLLLSLVITLYCKRGRK |
| | KLLYIFKQPFMRPVQTTQEEDGCSCRFPEEEEGGCELRVKFSRSA |
| | DAPAYQQGQNQLYNELNLGRREEYDVLDKRRGRDPEMGGKPR |
| | RKNPQEGLYNELQKDKMAEAYSEIGMKGERRRGKGHDGLYQG |
| | LSTATKDTYDALHMQALPPR (SEQ ID NO: 4) |
| CD70 CAR 4 | ATPAPKSCPERHYWAQGKLCCQMCEPGTFLVKDCDQHRKAAQ |
| | CDPCIPGVSFSPDHHTRPHCESCRHCNSGLLVRNCTITANAECAC |
| | RNGWQCRDKECTECDGGGSGGGSGGGSGGGSGGGSGGGSGGG |
| | SGGGSGGGSGGGSGGGSGGGSGGGSGGGSGGGSGGGSGGGSIL |
| | VIFSGMFLVFTLAGALFLKRGRKKLLYIFKQPFMRPVQTTQEED |
| | GCSCRFPEEEEGGCELRVKFSRSADAPAYQQGQNQLYNELNLGR |
| | REEYDVLDKRRGRDPEMGGKPRRKNPQEGLYNELQKDKMAEA |
| | YSEIGMKGERRRGKGHDGLYQGLSTATKDTYDALHMQALPPR |
| | (SEQ ID NO: 5) |
| CD70 CAR 5 | ATPAPKSCPERHYWAQGKLCCQMCEPGTFLVKDCDQHRKAAQ |
| | CDPCIPGVSFSPDHHTRPHCESCRHCNSGLLVRNCTITANAECAC |
| | RNGWQCRDKECTECDPLPNPSLTARSSLCSSDFIRILVIFSGMFLV |
| | FTLAGALFLKRGRKKLLYIFKQPFMRPVQTTQEEDGCSCRFPEEE |
| | EGGCELRVKFSRSADAPAYQQGQNQLYNELNLGRREEYDVLDK |
| | RRGRDPEMGGKPRRKNPQEGLYNELQKDKMAEAYSEIGMKGE |
| | RRRGKGHDGLYQGLSTATKDTYDALHMQALPPR (SEQ ID NO: |
| | 6) |
| CD70 CAR 6 | ATPAPKSCPERHYWAQGKLCCQMCEPGTFLVKDCDQHRKAAQ |
| | CDPCIPGVSFSPDHHTRPHCESCRHCNSGLLVRNCTITANAECAC |
| | RNGWQCRDKECTECDILVIFSGMFLVFTLAGALFLKRGRKKLLY |
| | IFKQPFMRPVQTTQEEDGCSCRFPEEEEGGCELRVKFSRSADAPA |
| | YQQGQNQLYNELNLGRREEYDVLDKRRGRDPEMGGKPRRKNP |
| | QEGLYNELQKDKMAEAYSEIGMKGERRRGKGHDGLYQGLSTA |
| | TKDTYDALHMQALPPR (SEQ ID NO: 7) |

### REFERENCES

1. Döhner H, Weisdorf D J, Bloomfield C D. Acute Myeloid Leukemia. New England Journal of Medicine. 2015; 373(12):1136-1152.
2. Guerra V A, DiNardo C, Konopleva M. Venetoclax-based therapies for acute myeloid leukemia. Best Pract Res Clin Haematol. 2019; 32(2):145-153.
3. Yarchoan M, Hopkins A, Jaffee E M. Tumor Mutational Burden and Response Rate to PD-1 Inhibition. N Engl J Med. 2017; 377(25):2500-2501.
4. Daver N G, Basu S, Garcia-Manero G, et al. Phase IB/II study of nivolumab with azacytidine (AZA) in patients (pts) with relapsed AML. Journal of Clinical Oncology. 2017; 35(15_suppl):7026-7026.
5. Lawrence M S, Stojanov P, Polak P, et al. Mutational heterogeneity in cancer and the search for new cancer-associated genes. Nature. 2013; 499(7457):214-218.
6. Papaemmanuil E, Gerstung M, Bullinger L, et al. Genomic Classification and Prognosis in Acute Myeloid Leukemia. New England Journal of Medicine. 2016; 374(23):2209-2221.
7. Pyzer A R, Stroopinsky D, Rosenblatt J, et al. Myeloid-Derived Suppressor Cells Are Expanded in Patients with AML and Are Dependent on MUC1 Expression. Blood. 2014; 124(21):226-226.
8. Alex A A, Tartour E, Gey A, et al. Myeloid Derived Suppressor Cells in Acute Leukemia and Its Association with Conventional Cytogenetic and Molecular Risk Factors. Blood. 2012; 120(21):1446-1446.
9. Williams P, Basu S, Garcia-Manero G, et al. The distribution of T-cell subsets and the expression of immune checkpoint receptors and ligands in patients with newly diagnosed and relapsed acute myeloid leukemia. Cancer. 2019; 125(9):1470-1481.
10. Schuster S J, Svoboda J, Chong E A, et al. Chimeric Antigen Receptor T Cells in Refractory B-Cell Lymphomas. New England Journal of Medicine. 2017; 377(26):2545-2554.
11. Neelapu S S, Locke F L, Bartlett N L, et al. Axicabtagene Ciloleucel CAR T-Cell Therapy in Refractory Large B-Cell Lymphoma. New England Journal of Medicine. 2017; 377(26):2531-2544.
12. Mardiros A, Dos Santos C, McDonald T, et al. T cells expressing CD123-specific chimeric antigen receptors exhibit specific cytolytic effector functions and antitumor effects against human acute myeloid leukemia. Blood. 2013; 122(18):3138-3148.
13. Luo Y, Chang L-J, Hu Y, Dong L, Wei G, Huang H. First-in-Man CD123-Specific Chimeric Antigen Receptor-Modified T Cells for the Treatment of Refractory Acute Myeloid Leukemia. Blood. 2015; 126(23):3778-3778.
14. Wang Q-s, Wang Y, Lv H-y, et al. Treatment of CD33-directed Chimeric Antigen Receptor-modified T Cells in One Patient With Relapsed and Refractory Acute Myeloid Leukemia. Molecular Therapy. 2015; 23(1):184-191.
15. Kim M Y, Yu K-R, Kenderian S S, et al. Genetic Inactivation of CD33 in Hematopoietic Stem Cells to Enable CAR T Cell Immunotherapy for Acute Myeloid Leukemia. Cell. 2018; 173(6):1439-1453.e1419.
16. Jetani H, Garcia-Cadenas I, Nerreter T, et al. CAR T-cells targeting FLT3 have potent activity against FLT3−ITD+ AML and act synergistically with the FLT3-inhibitor crenolanib. Leukemia. 2018; 32(5):1168-1179.
17. Hofmann S, Schubert M L, Wang L, et al. Chimeric Antigen Receptor (CAR) T Cell Therapy in Acute Myeloid Leukemia (AML). J Clin Med. 2019; 8(2).
18. Melao A. FDA Suspends UCART123 Trials After Patient Death. Immuno-Oncology News; 2017.
19. Perna F, Berman S H, Soni R K, et al. Integrating Proteomics and Transcriptomics for Systematic Combinatorial Chimeric Antigen Receptor Therapy of AML. Cancer Cell. 2017; 32(4):506-519.e505.
20. Riether C, Schürch C M, Bührer E D, et al. CD70/CD27 signaling promotes blast stemness and is a viable therapeutic target in acute myeloid leukemia. The Journal of Experimental Medicine. 2017; 214(2):359-380.
21. Ochsenbein A. Argx-110 Targeting CD70, in Combination with Azacitidine, Shows Favorable Safety Profile and Promising Anti-Leukemia Activity in Newly Diagnosed AML Patients in an Ongoing Phase 1/2 Clinical Trial. Abstract 2680. American Society of Hematology Annual Meeting. San Diego, Calif.; 2018.
22. Slaney C Y, Wang P, Darcy P K, Kershaw M H. CARs versus BiTEs: A Comparison between T Cell-Redirection Strategies for Cancer Treatment. Cancer Discov. 2018; 8(8):924-934.
23. Wang Q J, Yu Z, Hanada K I, et al. Preclinical Evaluation of Chimeric Antigen Receptors Targeting CD70-Expressing Cancers. Clin Cancer Res. 2017; 23(9):2267-2276.
24. Fry T J, Shah N N, Orentas R J, et al. CD22-targeted CAR T cells induce remission in B-ALL that is naive or resistant to CD19-targeted CAR immunotherapy. Nat Med. 2018; 24(1):20-28.
25. Ramakrishna S, Highfill SL, Walsh Z, et al. Modulation of Target Antigen Density Improves CAR T-cell Functionality and Persistence. Clinical Cancer Research. 2019; 25(17):5329-5341.
26. Pont MJ, Hill T, Cole G O, et al. gamma-secretase inhibition increases efficacy of BCMA-specific chimeric antigen receptor T cells in multiple myeloma. Blood. 2019.
27. Ossenkoppele G, Löwenberg B. How I treat the older patient with acute myeloid leukemia. Blood. 2015; 125(5):767-774.
28. Information NCfB. Azacitidine CID=9444. PubChem Database; 2020.
29. Kitajima S, Lee KL, Fujioka M, et al. Hypoxia-inducible factor-2 alpha up-regulates CD70 under hypoxia and enhances anchorage-independent growth and aggressiveness in cancer cells. Oncotarget. 2018; 9(27).
30. Fry T. Optimizing CAR T cell for AML:What Can we Learn FRom Experience in ALL. The Boston Society Acute Myeloid Leukemia Medical Symposium. Broad Institute of MIT, Boston, Mass.; 2019.
31. Marcucci G, Silverman L, Eller M, Lintz L, Beach C L. Bioavailability of azacitidine subcutaneous versus intravenous in patients with the myelodysplastic syndromes. J Clin Pharmacol. 2005; 45(5):597-602.
32. Bonifant C L, Szoor A, Tones D, et al. CD123-Engager T Cells as a Novel Immunotherapeutic for Acute Myeloid Leukemia. Molecular Therapy. 2016; 24(9):1615-1626.
33. Petrov J C, Wada M, Pinz K G, et al. Compound CAR T-cells as a double-pronged approach for treating acute myeloid leukemia. Leukemia. 2018; 32(6):1317-1326.
34. Duong M T, Collinson-Pautz M R, Morschl E, et al. Two-Dimensional Regulation of CAR-T Cell Therapy with Orthogonal Switches. Molecular Therapy—Oncolytics. 2019; 12:124-137.
35. Derissen E J B, Beijnen J H, Schellens J H M. Concise drug review: azacitidine and decitabine. The oncologist. 2013; 18(5):619-624.
36. Gill S I. How close are we to CAR T-cell therapy for AML? Best Practice & Research Clinical Haematology. 2019; 32(4):101104.
37. Ritchie D S, Neeson P J, Khot A, et al. Persistence and efficacy of second generation CAR T cell against the LeY antigen in acute myeloid leukemia. Mol Ther. 2013; 21(11):2122-2129.
38. Wang Q S, Wang Y, Lv H Y, et al. Treatment of CD33-directed chimeric antigen receptor-modified T cells in one patient with relapsed and refractory acute myeloid leukemia. Mol Ther. 2015; 23(1):184-191.
39. Cummins K D, Frey N, Nelson A M, et al. Treating Relapsed/Refractory (RR) AML with Biodegradable Anti-CD123 CAR Modified T Cells. Blood. 2017; 130 (Supplement 1):1359-1359.
40. Yao S, Jianlin C, Yarong L, et al. Donor-Derived CD123-Targeted CAR T Cell Serves as a RIC Regimen for Haploidentical Transplantation in a Patient With FUS-ERG+ AML. Frontiers in Oncology. 2019; 9(1358).
41. Budde E L. Remissions of Acute Myeloid Leukemia and Blastic Plasmacytoid Dendritic Cell Neoplasm Following Treatment with CD123-Specific CAR T Cells: A First-in-Human Clinical Trial. American Society of Hematology Annual Meeting. Atlanta, Ga. 2017.
42. Sallman D A, Brayer J, Sagatys E M, et al. NKG2D-based chimeric antigen receptor therapy induced remission in a relapsed/refractory acute myeloid leukemia patient. Haematologica. 2018:haematol. 2017.186742.
43. Baumeister S H, Murad J, Werner L, et al. Phase I Trial of Autologous CART Cells Targeting NKG2D Ligands in Patients with AML/MDS and Multiple Myeloma. Cancer Immunology Research. 2019; 7(1):100.
44. Gill S. Past Present and Future of CAR T cell Therapy for AML. American Society of Transplantation and Cellular Therapy Orlando, Fla.; 2020.
45. Ochsenbein A. Targeting CD70 with Cusatuzumab Eliminates Acute Myeloid Leukemia Stem Cells in Humans. American Society of Hematology. Orlando, Fla. 2019.
46. Turtle CJ, Hanafi L-A, Berger C, et al. CD19 CAR-T cells of defined CD4+:CD8+ composition in adult B cell ALL patients. The Journal of Clinical Investigation. 2016; 126(6):2123-2138.
47. Majzner R G, Mackall C L. Tumor Antigen Escape from CAR T-cell Therapy. Cancer Discovery. 2018; 8(10):1219.
48. Fraietta J A, Lacey S F, Orlando E J, et al. Determinants of response and resistance to CD19 chimeric antigen receptor (CAR) T cell therapy of chronic lymphocytic leukemia. Nature Medicine. 2018; 24(5):563-571.
49. Leick M, Maus M V. Wishing on a CAR: Understanding the Scope of Intrinsic T-cell Deficits in Patients with Cancer. Cancer Discovery. 2019; 9(4):466.
50. Das R K, Vernau L, Grupp S A, Barrett DM. Naïve T-cell Deficits at Diagnosis and after Chemotherapy Impair Cell Therapy Potential in Pediatric Cancers. Cancer Discovery. 2019; 9(4):492.
51. Pont M J, Hill T, Cole G O, et al. gamma-Secretase inhibition increases efficacy of BCMA-specific chimeric antigen receptor T cells in multiple myeloma. Blood. 2019; 134(19):1585-1597.
52. Riddell S. Integrating Targeted Therapies and Cell Based Therapies American Society of Transplantation and Cellular Therapy. Orlando, Fla. 2020.
53. Fraietta J A, Beckwith K A, Patel P R, et al. Ibrutinib enhances chimeric antigen receptor T-cell engraftment and efficacy in leukemia. Blood. 2016; 127(9):1117-1127.

All publications, patents, patent applications, publication, and database entries (e.g., sequence database entries) mentioned herein, e.g., in the Background, Summary, Detailed Description, Examples, and/or References sections, are hereby incorporated by reference in their entirety as if each individual publication, patent, patent application, publication, and database entry was specifically and individually incorporated herein by reference. In case of conflict, the present application, including any definitions herein, will control.

### EQUIVALENTS AND SCOPE

Those skilled in the art will recognize, or be able to ascertain using no more than routine experimentation, many equivalents of the embodiments described herein. The scope of the present disclosure is not intended to be limited to the above description, but rather is as set forth in the appended claims.

Articles such as “a,” “an,” and “the” may mean one or more than one unless indicated to the contrary or otherwise evident from the context. Claims or descriptions that include “or” between two or more members of a group are considered satisfied if one, more than one, or all of the group members are present, unless indicated to the contrary or otherwise evident from the context. The disclosure of a group that includes “or” between two or more group members provides embodiments in which exactly one member of the group is present, embodiments in which more than one members of the group are present, and embodiments in which all of the group members are present. For purposes of brevity those embodiments have not been individually spelled out herein, but it will be understood that each of these embodiments is provided herein and may be specifically claimed or disclaimed.

It is to be understood that the disclosure encompasses all variations, combinations, and permutations in which one or more limitation, element, clause, or descriptive term, from one or more of the claims or from one or more relevant portion of the description, is introduced into another claim. For example, a claim that is dependent on another claim can be modified to include one or more of the limitations found in any other claim that is dependent on the same base claim. Furthermore, where the claims recite a composition, it is to be understood that methods of making or using the composition according to any of the methods of making or using disclosed herein or according to methods known in the art, if any, are included, unless otherwise indicated or unless it would be evident to one of ordinary skill in the art that a contradiction or inconsistency would arise.

Where elements are presented as lists, e.g., in Markush group format, it is to be understood that every possible subgroup of the elements is also disclosed, and that any element or subgroup of elements can be removed from the group. It is also noted that the term “comprising” is intended to be open and permits the inclusion of additional elements or steps. It should be understood that, in general, where an embodiment, product, or method is referred to as comprising particular elements, features, or steps, embodiments, products, or methods that consist, or consist essentially of, such elements, features, or steps, are provided as well. For purposes of brevity those embodiments have not been individually spelled out herein, but it will be understood that each of these embodiments is provided herein and may be specifically claimed or disclaimed.

Where ranges are given, endpoints are included. Furthermore, it is to be understood that unless otherwise indicated or otherwise evident from the context and/or the understanding of one of ordinary skill in the art, values that are expressed as ranges can assume any specific value within the stated ranges in some embodiments, to the tenth of the unit of the lower limit of the range, unless the context clearly dictates otherwise. For purposes of brevity, the values in each range have not been individually spelled out herein, but it will be understood that each of these values is provided herein and may be specifically claimed or disclaimed. It is also to be understood that unless otherwise indicated or otherwise evident from the context and/or the understanding of one of ordinary skill in the art, values expressed as ranges can assume any subrange within the given range, wherein the endpoints of the subrange are expressed to the same degree of accuracy as the tenth of the unit of the lower limit of the range.

Where websites are provided, URL addresses are provided as non-browser-executable codes, with periods of the respective web address in parentheses. The actual web addresses do not contain the parentheses.

In addition, it is to be understood that any particular embodiment of the present disclosure may be explicitly excluded from any one or more of the claims. Where ranges are given, any value within the range may explicitly be excluded from any one or more of the claims. Any embodiment, element, feature, application, or aspect of the compositions and/or methods of the disclosure, can be excluded from any one or more claims. For purposes of brevity, all of the embodiments in which one or more elements, features, purposes, or aspects is excluded are not set forth explicitly herein.

What is claimed is:

1. A chimeric antigen receptor (CAR) comprising:
(i) an extracellular target binding domain comprising a polypeptide that binds CD70;
(ii) a transmembrane domain; and
(iii) an intracellular signaling domain.

2. The CAR of claim 1, wherein the polypeptide comprises a CD70-binding domain of CD27.

3. The CAR of claim 1 or claim 2, wherein the polypeptide comprises the extracellular domain of CD27.

4. The CAR of any one of claims 1-3, wherein the polypeptide comprises an amino acid sequence that is at least 80% identical to the amino acid sequence of SEQ ID NO: 1.

5. The CAR of claim 4, wherein the polypeptide comprises the amino acid sequence of any one of SEQ ID NO: 1.

6. The CAR of claim 1, wherein the polypeptide comprises an anti-CD70 antibody, optionally an scFv.

7. The CAR of any one of claims 1-6, wherein the transmembrane domain is the transmembrane domain of CD27.

8. The CAR of any one of claims 1-7, wherein the intracellular signaling domain comprises (i) an ITAM-containing signaling domains and/or (ii) one or more signaling domains from one or more co-stimulatory proteins or cytokine receptors.

9. The CAR of any one of claims 1-8, wherein the intracellular signaling domain comprises a CD3γ, CD3ε, CD3δ or CD3ζ.

10. The CAR of anyone of claims 1-9, wherein the intracellular signaling domain comprises CD3.

11. The CAR of any one of claims 8-10, wherein the costimulatory domain comprises CD28, 41BB, 2B4, KIR, OX40, ICOS, MYD88, IL2 receptor, or SynNotch.

12. The CAR of any one of claims 8-11, wherein the costimulatory domain comprises 41BB.

13. The CAR of any one of claims 1-12, wherein the CAR comprises an amino acid sequence that is at least 80% identical to the amino acid sequence of any one of SEQ ID NOs: 2-7.

14. The CAR of claim 13, wherein the CAR comprises the amino acid sequence of any one of SEQ ID NO: 2-7.

15. The CAR of any one of claims 1-14, wherein the extracellular target binding domain further comprises a signal peptide, optionally wherein the signal peptide comprises a CD27 signal peptide.

16. A nucleic acid comprising a nucleotide sequence encoding the CAR of any one of claims 1-15.

17. The nucleic acid of claim 16, wherein the nucleotide is operably linked to a promoter.

18. The nucleic acid of claim 17 wherein the promoter is an EF1-alpha promoter.

19. A vector comprising the nucleic acid of any one of claims 16-18.

20. The vector of claim 18, wherein the vector is a retroviral vector, a lentiviral vector or an AAV.

21. An engineered immune cell comprising the CAR of any one of claims 1-15.

22. The engineered immune cell of claim 21, wherein the immune cell is a T-cell, a NK cell, a dendritic cell, a macrophage, a B cell, a neutrophil, an eosinophil, a basophil, a mast cell, a myeloid derived suppressor cell, a mesenchymal stem cell, a precursor thereof, or a combination.

23. The engineered immune cell of claim 21 or claim 22, wherein the immune cell is a T-Cell.

24. The engineered immune cell of any one of claims 21-23, wherein immune cell is autologous or allogeneic.

25. A method comprising administering to a subject the engineered immune cell of any one of claims 21-24.

26. A method of treating a cancer expressing CD70, the method comprising administering to a subject in need thereof an effective amount of the engineered immune cell of any one of claims 21-24.

27. A method of treating a cancer expressing CD70, the method comprising administering to a subject in need thereof a therapeutically effective amount of the engineered immune cell of any one of claims 21-24 and an effective amount of an agent that enhances expression of CD70 in the cancer.

28. The method of claim 27, wherein the agent results in hypomethylation of CD-70 encoding gene in the cancer.

29. The method of claim 28, wherein the agent is azacitidine or decitabine.

30. The method of any one of claims 27-29, wherein the engineered immune cell and the agent are administered simultaneously.

31. The method of claim 30, wherein the engineered immune cell and the agent are formulated in a composition.

32. The method of claim 31, wherein the agent is azacitidine having a concentration of 10 μM or less in the composition.

33. The method of any one of claims 27-29, wherein the engineered immune cell and the agent are administered sequentially.

34. The method of claim 33, wherein the agent is administered before the engineered immune cell is administered.

35. The method of claim 34, further comprising waiting a period of time between administering the agent and administering the engineered immune cell.

36. The method of any one of claims 25-35, wherein the subject is human

37. The method of any one of claims 25-36, wherein the administering is via infusion.

38. The method of any one of claims 26-37, wherein the cancer is a myeloid cancer.

39. The method of any one of claims 26-38, wherein the cancer is acute myeloid leukemia.
